(19) United States
(12) Patent Application Publication
(10) Pub. No.: US 2002/0177566 A1
(43) Pub. Date: Nov. 28, 2002

(54) Nucleic acid sequences associated with baldness

(76) Inventors: David Pritchard, Seattle, WA (US); Glenna Burmer, Seattle, WA (US); Joseph Brown, Seattle, WA (US); Vasiliki Demas, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/825,096

(22) Filed: Apr. 2, 2001

Related U.S. Application Data
(60) Provisional application No. 60/199,745, filed on Apr. 25, 2000.

(51) Int. Cl. A61K 48/00; C12Q 1/68; G01N 33/567; A61K 7/06

(52) U.S. Cl. 514/44; 435/6; 435/7.21; 424/70.1

(57) ABSTRACT

This invention relates to the discovery of nucleic acids and proteins associated with baldness and/or hair loss. The identification of these baldness-associated nucleic acids and proteins have uses in predicting the propensity for baldness of an individual and/or in determining the likelihood of baldness in an individual experiencing hair loss. In addition, the nucleic acids of the invention can be used can be used for gene therapy for delaying or stopping the progression of baldness, and/or for reversing baldness.

### CROSS-REFERENCES TO RELATED APPLICATIONS

The present application claims priority to U.S. Ser. No. 60/199,745, filed Apr. 25, 2000, herein incorporated by reference in its entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Hair loss can be caused by illness (e.g., fever, thyroid function imbalance, skin disease, infection or autoimmune disorders), or can be due to extrinsic factors, such as medical treatments (e.g., chemotherapy and radiotherapy), dietary imbalances or stress, as well as to pregnancy and intrinsic factors (e.g., genetic factors, hormone production, hormonal imbalances, aging, etc.). Hair loss due to extrinsic factors, pregnancy or curable diseases or imbalances generally stops when normal condition is restored, and the hair grows back. In contrast, hair loss due to intrinsic factors is often irreversible and results in partial or complete baldness.

With age, both men and women lose hair density and this gradual thinning of the hair results in baldness in a number of cases. Baldness affects a large proportion of the population, since about 35% of men begin to bald by the time they are 35 years old, and about two-thirds are either bald or have a balding pattern by age 60. Although inherited baldness affects more men than women, the incidence of baldness in women is significant, since it amounts to a third or half of that in men before menopause, and increases greatly after that. Baldness is neither physically disabling nor a life-threatening disease, and is only of cosmetic importance, but it may profoundly affect self-esteem and/or cause psychological stress and anxiety.

Hair consists of a soft bulb, called the root, and a shaft. The root and a section of the shaft below the skin surface lie in a follicle sac. The bottom of the follicle sac projects the papilla which contains an artery that nourishes the root. The hair grows by forming new cells at the base of the root, which is a highly proliferative cell population. The cells form around the nourishing papilla, as the old ones are pushed away, die and become part of the shaft. Human scalp hair usually grows one-half inch per month for two to four years. The shaft then falls off and is replaced by a new shaft. When the old shaft falls off, the papilla becomes active again and new hair appears.

Changes that contribute to the development of baldness include alterations in the growth cycle of hair. Hair typically progresses through cycles comprising three phases: anagen (active hair growth), catagen (transition phase), and telogen (resting phase during which the hair shaft is shed prior to new growth). As baldness progresses, there is a shift in the percentages of hair follicles in each phase, with the majority shifting from anagen to telogen. The size of hair follicles is also known to decrease while the total number remains relatively constant. Baldness results when the old shaft is no longer replaced. In most cases, the hair follicle remains alive and the potential for hair re-growth is preserved.

Despite many efforts, the role of hormones in regulating the hair cycle is not yet thoroughly understood. The role of androgens is particularly puzzling. While in most body sites androgens stimulate hair growth by prolonging the growth phase and increasing follicle size, hair growth on the scalp does not require androgens. Paradoxically, androgens (e.g., testosterone) are believed to be necessary for balding on the scalp in genetically predisposed individuals where there is a progressive decline in the duration of anagen and in hair follicle size. However, only a proportion of men develop baldness and there is no difference in circulating testosterone levels between bald and non-bald men.

At present, there is no clear explanation to the development of baldness and no reasonable hypothesis on which to either predict a pre-disposition to hair loss or base a systematic search for new and improved treatments. Development of new effective treatments for baldness has thus been limited. Current treatments include, for example, the administration of nitroxides (e.g., Minoxidil, Nicorandil, etc.), antiandrogens (e.g., Proscar, Cyoctal, spironolactone, etc.), superoxide dismutase mimetics, etc. While progress has been made in the stimulation of hair-growth by drug treatment, none of the available treatments is completely satisfactory and most of them have undesirable associated side effects. In addition, in a number of cases, hair loss resumes if the treatment is stopped. Alternative solutions include hair surgery, e.g., hair transplantation, scalp reduction, etc. Such procedures are time-consuming, invasive, expensive and can only be used in certain cases.

In view of the foregoing, it is readily apparent that there is a great need in the art for new and effective treatments for baldness and hair loss, as well as for tools for predicting the propensity for balding of a subject which would allow to prevent the development of baldness. The present invention addresses these and other needs.

### SUMMARY OF THE INVENTION

The present invention provides isolated nucleic acids and proteins associated with baldness and hair-loss. The sequences of the present invention associated with baldness can be used to determine the propensity of an individual for baldness as well as for determining the likelihood of developing baldness of an individual experiencing hair-loss. Such sequences can also be targeted and their level of expression altered by, for example, gene therapy methods (e.g., by altering the subject sequences). Such methods can be used, for example, to slow or stop hair-loss, to stimulate hair follicle activity, to stimulate hair growth and/or to reverse baldness. They can also be used to determine the activity and size of hair follicles in a individual.

As such, the present invention provides a method for predicting the propensity for baldness, the method comprising detecting the overexpression or the underexpression of a baldness-associated molecule of interest according to Table 1 in a subject, wherein the overexpression or the underexpression of the molecule is indicative of a propensity for baldness. In some embodiments, overexpression of the baldness-associated molecule of interest is indicative of a propensity for baldness and the molecule of interest is overexpressed in the subject. In other embodiments, underexpression of the baldness-associated molecule of interest is indicative of a propensity for baldness and the molecule of interest is underexpressed in the subject. In one embodiment, the baldness-associated molecule of interest is detected by detecting an mRNA encoding the molecule. In another embodiment, the baldness-associated molecule is detected in an immunoassay.

In another aspect, the present invention provides a method for identifying a modulator of hair loss, the method comprising culturing a cell in the presence of a modulator to form a first cell culture, contacting RNA or cDNA from the first cell culture with a probe which comprises a polynucleotide sequence that encodes a baldness-associated protein of interest, and determining whether the amount of probe that hybridizes to the RNA or cDNA from the first cell culture is increased or decreased relative to the amount of the probe that hybridizes to RNA or cDNA from a second cell culture grown in the absence of the modulator. In one embodiment, the polynucleotide sequences associated with baldness are selected from the group consisting of the sequences set forth in Table 1. In another embodiment, the first and second cell cultures are obtained from a scalp cell.

The present invention also provides a method for inhibiting the development of baldness, the method comprising introducing into a cell a baldness-associated molecule, wherein underexpression of the baldness-associated molecule is indicative of a propensity for baldness. In one embodiment, a nucleic acid encoding a baldness-associated protein is introduced into the cell. In another embodiment, the baldness-associated molecule introduced into the cell is a protein. In some embodiments, the baldness-associated molecule is selected from the group consisting of the sequences set forth in Table 1. The present invention also provides a method for reversing baldness, the method comprising the steps of introducing into a cell a baldness-associated molecule, wherein underexpression of the baldness-associated molecule is indicative of a propensity for baldness. The baldness-associated molecule introduced into the cell may be a nucleic acid encoding a baldness-associated protein or a protein. In one embodiment, the baldness-associated molecule is selected from the group consisting of the sequences set forth in Table 1.

The present invention further provides a method for inhibiting the development of baldness, the method comprising inhibiting in a cell a baldness-associated molecule, wherein overexpression of the baldness-associated molecule is indicative of a propensity for baldness. The baldness-associated molecule may be inhibited using an antisense polynucleotide or an antibody that specifically binds to the baldness-associated molecule. In some embodiments, the baldness-associated molecule is selected from the group consisting of the sequences set forth in Table 1. In addition, the present invention provides a method for reversing baldness, the method comprising inhibiting in a cell a baldness-associated molecule according to Table 1, wherein overexpression of the baldness-associated molecule is indicative of a propensity for baldness. Again, the baldness-associated molecule may be inhibited using either an antibody that specifically binds to the baldness-associated molecule or an antisense polynucleotide. In some embodiments, the baldness-associated molecule is selected from the group consisting of the sequences set forth in Table 1.

In yet another aspect, the present invention provides a method for inhibiting the development of baldness in a patient in need thereof, the method comprising administering to the patient a compound that modulates hair loss. In addition, the present invention provides a method for reversing baldness in a patient, the method comprising administering to the patient a compound that modulates hair loss.

The present invention is also directed to a kit for detecting whether a scalp cell is becoming dormant, the kit comprising a probe which comprises a polynucleotide sequence associated with baldness, and a label for detecting the presence of the probe. In one embodiment, the polynucleotide associated with baldness is selected from the group consisting of the sequences set forth in Table 1.

The present invention further provides a cosmetic composition for inhibiting the development of baldness in a patient, the cosmetic composition comprising a compound that modulates hair loss. The cosmetic composition may be in a form including, but not limited to, shampoos, conditioners, lotions, sprays, ointments, oils, and gels. In addition, the present invention provides a cosmetic composition for reversing baldness. Again, the composition may be in a form including, but not limited to shampoos, conditioners, lotions, sprays, ointments, oils, and gels.

### BRIEF DESCRIPTION OF THE DRAWINGS

Not applicable.

### DESCRIPTION OF THE SPECIFIC EMBODIMENTS

I. INTRODUCTION

The present invention provides nucleic acids and proteins that are useful for treating baldness and for determining the propensity for baldness, and/or of alopecia, hair loss, dormant and/or miniature hair follicles.

Host cells, vectors and probes are described, as are antibodies to the proteins and uses of the proteins as antigens. The present invention provides methods for obtaining and expressing nucleic acids, methods for purifying gene products, methods for detecting and quantifying the expression and quality of the gene product (e.g., proteins), and uses for both the nucleic acids and the gene products. The probes and antibodies are useful for predicting the propensity for baldness and for determining the likelihood to develop baldness of an individual experiencing hair loss. In addition, the nucleic acids, antisense polynucleotides and polypeptides of the invention are useful for gene therapy applications.

The present invention also provides methods for screening for modulators of baldness. Such modulators are useful for preventing and/or reversing baldness.

This invention relies on routine techniques in the field of recombinant genetics. A basic text disclosing the general methods of use in this invention is Sambrook et al., Molecular Cloning, A Laboratory Manual, Cold Spring Harbor Publish., Cold Spring Harbor, N.Y. 2nd ed. (1989); and Kriegler, Gene Transfer and Expression: A Laboratory Manual, Freeman, N.Y. (1990). Unless otherwise stated all enzymes are used in accordance with the manufacturer's instructions.

II. DEFINITIONS

In the context of the present invention, “baldness” encompasses the complete or partial loss of hair and a variety of different types of alopecia (e.g., alopecia areata, alopecia totalis, alopecia universalis, alopecia diffusa, alopecia partialis and androgenic alopecia) due to intrinsic factors (e.g., aging, hormone production and/or hormonal imbalances, pregnancy, etc.) or genetic factors, as well as disease- or extrinsic factors-related hair loss (e.g., thyroid function imbalance, autoimmune disorders, stress, vitamin deficiency and/or other dietary imbalances, chemotherapy, radiotherapy or other treatments, etc.). “Baldness” of the scalp is characterized by, e.g., loss of activity and/or miniaturization of hair follicles, hair loss, slowing of hair growth, thinning of the hair, appearance of shorter and weaker hairs, as well as any of a number of characteristic structural and/or molecular features. In the context of the present invention, “baldness” refers to all the stages of the process, e.g., receding hairline, thinning of hair, loss of hair at the crown of the head, hair-loss in a typical “M-shaped” pattern which eventually results in a loss of hair over the top of the head, complete hair loss, etc.

The term “transitional region” refers to those regions of the scalp of a subject experiencing hair loss and/or developing baldness that are at an intermediate state in the process. In addition, the term “transitional” may also refer to an individual who is developing baldness. In such “transitional regions” or “transitional individuals” hair loss is important but hair follicles retain some activity and hair is still present (although it may be significantly thinner).

“Baldness-associated” refers to the relationship of a nucleic acid and its expression, or lack thereof, or a protein and its level or activity, or lack thereof, to the onset, propensity and/or progression of hair loss, alopecia or baldness in a subject. For example, the propensity for hair loss or baldness can be associated with expression of a particular gene that is not expressed, or is expressed at a lower level, in a tissue of interest in an individual having no propensity for baldness (or in a non-bald individual or in a non-bald region of the scalp). Such a gene may also be expressed in a “transitional” individual or in a “transitional region of the scalp,” although expression may be at a lower level than in a bald individual or in a bald region of the scalp. Conversely, a baldness-associated gene, can be one that is not expressed or is expressed at a lower level in the scalp of an individual with a propensity for baldness, in a bald individual or in a bald region of the scalp than it is expressed in the scalp of a subject having no propensity for baldness, in a non-bald individual, or in a non-bald region of the scalp. Such a gene may also not be expressed or may be expressed at a lower level in a “transitional” individual or in a “transitional region of the scalp” than in a subject having no propensity for baldness, in a non-bald individual, or in a non-bald region of the scalp. A “baldness associated molecule” therefore refers to a baldness-associated nucleic acid or the protein that it encodes.

“Dormant hair follicles” refers to those hair follicles which are inactive and fail to grow new hairs. “Dormant hair follicles” are often miniaturized. Similarly, in the context of the present invention, “dormant scalp cells” refers to those cells from the scalp that show a decrease or arrest in growth, proliferation and/or activity. The appearance “dormant hair follicles” and/or “dormant scalp cells” in an individual may result in diminished hair growth, thinning of hair, shorter and/or weaker hairs, hair loss, baldness, etc.

“Amplification primers” are oligonucleotides comprising either natural or analog nucleotides that can serve as the basis for the amplification of a selected nucleic acid sequence. They include, for example, both polymerase chain reaction primers and ligase chain reaction oligonucleotides.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

“Biological samples” refers to any tissue or liquid sample having genomic DNA or other nucleic acids (e.g., mRNA) or proteins. It refers to samples of cells or tissue from a individual having no propensity for baldness, from a non-bald individual, from a non-bald region of the scalp, as well as samples of cells or tissue from a bald individual, from a bald region of the scalp or from a individual having a propensity for baldness. Samples of cells or tissue may also be from a “transitional individual” or from a “transitional region” of the scalp.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

As used herein a “nucleic acid probe” is defined as a nucleic acid capable of binding to a target nucleic acid (e.g., a nucleic acid associated with baldness) of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers (Tetrahedron Lett. 22:1859-1862 (1981)), or by the triester method according to Matteucci, et al. (J. Am. Chem. Soc. 103:3185 (1981)). A double stranded fragment may then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals or hydrogen bonds to a label such that the presence of the probe may be determined by detecting the presence of the label bound to the probe.

The phrase “a nucleic acid sequence encoding” refers to a nucleic acid which contains sequence information for a structural RNA such as rRNA, a tRNA, or the primary amino acid sequence of a specific protein or peptide, or a binding site for a transacting regulatory agent. This phrase specifically encompasses degenerate codons (i.e., different codons which encode a single amino acid) of the native sequence or sequences which may be introduced to conform with codon preference in a specific host cell.

“Stringent hybridization conditions” and “stringent hybridization wash conditions” in the context of nucleic acid hybridization experiments, such as Southern and northern hybridizations, are sequence dependent, and are different under different environmental parameters. Longer sequences hybridize specifically at higher temperatures. An extensive guide to the hybridization of nucleic acids is found in Tijssen Laboratory Techniques in Biochemistry and Molecular Biology—Hybridization with Nucleic Acid Probes, part I, chapter 2 “Overview of principles of hybridization and the strategy of nucleic acid probe assays,” Elsevier, NY (1993). Generally, highly stringent hybridization and wash conditions are selected to be about 5° C. lower than the thermal melting point (Tₘ) for the specific sequence at a defined ionic strength and pH. Typically, under “stringent conditions,” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al., supra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein (see, Harlow and Lane Antibodies, A Laboratory Manual, Cold Spring Harbor Publications, NY (1988) for a description of immunoassay formats and conditions that can be used to determine specific immunoreactivity). Typically, a specific or selective reaction will be at least twice the background signal or noise and more typically more than 10 to 100 times background.

III. DETECTION OF GENE EXPRESSION AND GENOMIC ANALYSIS OF BALDNESS-ASSOCIATED PROTEINS

The polynucleotides and polypeptides of the present invention can be employed as research reagents and materials for the discovery of treatments and diagnostics to human disease. It will be readily apparent to those of skill in the art that although the following discussion is directed to methods for detecting nucleic acids associated with baldness, similar methods can be used to detect nucleic acids associated with, e.g., hair loss, loss of activity and/or miniaturization of hair follicles, loss of activity, growth and/or proliferative potential of scalp cells, slowing of hair growth, thinning of hair, receding hairline, appearance of shorter and/or weaker hairs, etc.

As should be apparent to those of skill in the art, the invention is the identification of baldness-associated genes and the discovery that multiple nucleic acids are associated with baldness. Accordingly, the present invention also includes methods for detecting the presence, alteration or absence of baldness-associated nucleic acids (e.g., DNA or RNA) in a physiological specimen in order to determine, for example, the health of hair follicle or scalp cells in vitro, or ex vivo and their level of activity, i.e., proliferation state or not, and the genotype and risk of hair loss or baldness associated with mutations created in non-baldness sequences. Although any tissue having hair follicle cells bearing the genome of an individual, or RNA associated with baldness, can be used, the most convenient specimen will be scalp or hair follicle samples. It is also possible and preferred in some circumstances to conduct assays on cells that are isolated under microscopic visualization. A particularly useful method is the microdissection technique described in WO 95/23960. The cells isolated by microscopic visualization can be used in any of the assays described herein including both genomic and immunological based assays.

This invention provides methods of genotyping family members in which relatives are diagnosed with, e.g., partial or complete baldness, premature baldness, thinning hair, androgenic alopecia, etc. Conventional methods of genotyping are provided herein.

The invention provides methods for detecting whether a cell, and in particular a hair follicle or a scalp cell, is in a dormant state, is losing activity, and/or is growing and/or dividing at a slower rate. The methods typically comprise contacting RNA from the cell with a probe which comprises a polynucleotide sequence associated with baldness and determining whether the amount of the probe which hybridizes to the RNA is increased or decreased relative to the amount of the probe which hybridizes to RNA from a hair follicle cell from a non-bald individual, from a non-bald region of the scalp or from an individual having no propensity for baldness. The assays are useful for detecting cell degeneration associated with, for example, baldness.

The probes are capable of binding to a target nucleic acid (e.g., a nucleic acid associated with baldness). By assaying for the presence or absence of the probe, one can detect the presence or absence of the target nucleic acid in a sample. Preferably, non-hybridizing probe and target nucleic acids are removed (e.g., by washing) prior to detecting the presence of the probe.

A variety of methods of specific DNA and RNA measurement using nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a baldness-associated gene of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins “Nucleic Acid Hybridization, A Practical Approach,” IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immunoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of the methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands which bind to labeled antibodies, fluorophores, chemi-luminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N.Y. (1997); and in Haugland Handbook of Fluorescent Probes and Research Chemicals, a combined handbook and catalogue Published by Molecular Probes, Inc. (1996).

In general, a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

Most typically, the amount of, for example, a baldness-associated RNA is measured by quantitating the amount of label fixed to the solid support by binding of the detection reagent. Typically, the presence of a modulator during incubation will increase or decrease the amount of label fixed to the solid support relative to a control incubation which does not comprise the modulator, or as compared to a baseline established for a particular reaction type. Means of detecting and quantitating labels are well known to those of skill in the art.

In preferred embodiments, the target nucleic acid or the probe is immobilized on a solid support. Solid supports suitable for use in the assays of the invention are known to those of skill in the art. As used herein, a solid support is a matrix of material in a substantially fixed arrangement. Exemplar solid supports include glasses, plastics, polymers, metals, metalloids, ceramics, organics, etc. Solid supports can be flat or planar, or can have substantially different conformations. For example, the substrate can exist as particles, beads, strands, precipitates, gels, sheets, tubing, spheres, containers, capillaries, pads, slices, films, plates, dipsticks, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham, Mass.), Ciba Corning (Medfield, Mass.), Bangs Laboratories (Carmel, Ind.), and BioQuest, Inc. (Atkinson, N.H.). The substrate is chosen to maximize signal to noise ratios, primarily to minimize background binding, for ease of washing and cost.

A variety of automated solid-phase assay techniques are also appropriate. For instance, very large scale immobilized polymer arrays (VLSIPS™), available from Affymetrix, Inc. (Santa Clara, Calif.) can be used to detect changes in expression levels of a plurality of baldness-associated nucleic acids simultaneously (see, Tijssen, supra.; Fodor et al. Science, 251:767-777 (1991); Sheldon et al. Clinical Chemistry 39(4):718-719 (1993); and Kozal et al. Nature Medicine 2(7):753-759 (1996)). Thus, in one embodiment, the invention provides methods of detecting the expression levels of baldness-associated nucleic acids in which nucleic acids (e.g., RNA from a cell culture) are hybridized to an array of nucleic acids that are known to be associated with baldness. For example, in the assay described supra, oligonucleotides which hybridize to a plurality of baldness-associated nucleic acids are optionally synthesized on a DNA chip (such chips are available from Affymetrix) and the RNA from a biological sample, such as a cell culture, is hybridized to the chip for simultaneous analysis of multiple baldness-associated nucleic acids. The baldness-associated nucleic acids that are present in the sample which is assayed are detected at specific positions on the chip.

Detection can be accomplished, for example, by using a labeled detection moiety that binds specifically to duplex nucleic acids (e.g., an antibody that is specific for RNA-DNA duplexes). One preferred example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies which are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include, but are not limited to, the selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through the use of a nucleic acid amplification system which multiplies the target nucleic acid being detected. Examples of such systems include the polymerase chain reaction (PCR) system and the ligase chain reaction (LCR) system. Other methods recently described in the art are the nucleic acid sequence based amplification (NASBAΘ, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation.

One embodiment is the use of allelic specific amplifications. In the case of PCR, the amplification primers are designed to bind to a portion of, for example, a gene encoding a baldness-associated protein, but the terminal base at the 3′ end is used to discriminate between the mutant and wild-type forms of the hair loss-associated protein gene. If the terminal base matches the point mutation or the wild-type, polymerase dependent three prime extension can proceed and an amplification product is detected. This method for detecting point mutations or polymorphisms is described in detail by Sommer et al. in Mayo Clin. Proc. 64:1361-1372 (1989). By using appropriate controls, one can develop a kit having both positive and negative amplification products. The products can be detected using specific probes or by simply detecting their presence or absence. A variation of the PCR method uses LCR where the point of discrimination, i.e., either the point mutation or the wild-type bases, fall between the LCR oligonucleotides. The ligation of the oligonucleotides becomes the means for discriminating between the mutant and wild-type forms of the baldness-associated protein gene.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the scalp or hair follicle cells, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

IV. IMMUNOLOGICAL DETECTION OF A BALDNESS-ASSOCIATED PROTEIN

In addition to the detection of the subject protein gene expression using nucleic acid hybridization technology, one can also use immunoassays to detect the protein itself. Immunoassays can be used to qualitatively or quantitatively analyze the proteins of interest. A general overview of the applicable technology can be found in Harlow and Lane, Antibodies: A Laboratory Manual, Cold Spring Harbor Pubs., NY (1988). Although the following discussion is directed to methods for detecting target proteins associated with baldness similar methods can be used to detect target proteins associated with, e.g., hair loss, loss of activity and/or miniaturization of hair follicles, slowing of hair growth, thinning of hair, receding hairline, appearance of shorter and/or weaker hairs, etc.

A. Antibodies to Target Proteins

Methods for producing polyclonal and monoclonal antibodies that react specifically with a protein of interest are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross-reactivity against non-baldness-associated proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and, most preferably, at about 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described infra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to the baldness-associated protein of interest. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including, e.g., injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., supra.

Once target protein specific antibodies are available, the protein can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum which was raised to the protein partially encoded by a sequence described herein or a fragment thereof. This antiserum is selected to have low cross-reactivity against non-baldness-associated proteins and any such cross-reactivity is removed by immunoabsorption prior to use in the immunoassay.

In order to produce antisera for use in an immunoassay, the baldness-associated protein of interest or a fragment thereof, for example, is isolated as described herein. For example, recombinant protein is produced in a transformed cell line. An inbred strain of mice, such as Balb/c, is immunized with the protein or a peptide using a standard adjuvant, such as Freund's adjuvant, and a standard mouse immunization protocol. Alternatively, a synthetic peptide derived from the sequences disclosed herein and conjugated to a carrier protein can be used as an immunogen. Polyclonal sera are collected and titered against the immunogen protein in an immunoassay, such as, for example, a solid phase immunoassay with the immunogen immobilized on a solid support. Polyclonal antisera with a titer of 10⁴ or greater are selected and tested for their cross-reactivity against non-baldness-associated proteins, using a competitive binding immunoassay such as the one described in Harlow and Lane, supra, at pages 570-573 and below.

B. Immunological Binding Assays

In a preferred embodiment, a protein of interest is detected and/or quantified using any of a number of well known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites & Terr, supra. Immunological binding assays (or immunoassays) typically utilize a “capture agent” to specifically bind to and often immobilize the analyte (e.g., the baldness-associated protein or antigenic subsequence thereof). The capture agent is a moiety that specifically binds to the analyte. In a preferred embodiment, the capture agent is an antibody that specifically binds, for example, the baldness-associated protein of interest. The antibody (e.g., anti-baldness-associated protein antibody) may be produced by any of a number of means well known to those of skill in the art and as described above.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled baldness-associated protein polypeptide or a labeled anti-baldness-associated protein antibody. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins of interest from tissue samples may be either competitive or noncompetitive. Noncompetitive immunoassays are assays in which the amount of captured analyte (in this case the protein) is directly measured. In one preferred “sandwich” assay, for example, the capture agent (e.g., anti-baldness-associated protein antibodies) can be bound directly to a solid substrate where it is immobilized. These immobilized antibodies then capture the baldness-associated protein present in the test sample. The baldness-associated protein thus immobilized is then bound by a labeling agent, such as a second anti-baldness-associated protein antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

2. Competitive Assay Formats

In competitive assays, the amount of target protein (analyte) present in the sample is measured indirectly by measuring the amount of an added (exogenous) analyte (e.g., the baldness-associated protein of interest) displaced (or competed away) from a capture agent (anti-baldness-associated protein antibody) by the analyte present in the sample. In one competitive assay, a known amount of, in this case, the protein of interest is added to the sample and the sample is then contacted with a capture agent, in this case an antibody that specifically binds to the baldness-associated protein. The amount of baldness-associated protein bound to the antibody is inversely proportional to the concentration of baldness-associated protein present in the sample. In a preferred embodiment, the antibody is immobilized on a solid substrate. The amount of the baldness-associated protein bound to the antibody may be determined either by measuring the amount of subject protein present in a baldness-associated protein/antibody complex or, alternatively, by measuring the amount of remaining uncomplexed protein. The amount of baldness-associated protein may be detected by providing a labeled baldness-associated protein molecule.

A hapten inhibition assay is another preferred competitive assay. In this assay, a known analyte, in this case the target protein, is immobilized on a solid substrate. A known amount of anti-baldness-associated protein antibody is added to the sample, and the sample is then contacted with the immobilized target. In this case, the amount of anti-baldness-associated protein antibody bound to the immobilized baldness-associated protein is inversely proportional to the amount of baldness-associated protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Immunoassays in the competitive binding format can be used for cross-reactivity determinations. For example, a protein encoded by the sequences described herein can be immobilized on a solid support. Proteins are added to the assay which compete with the binding of the antisera to the immobilized antigen. The ability of the above proteins to compete with the binding of the antisera to the immobilized protein is compared to that of a protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologues.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a preferred embodiment, Western blot (immunoblot) analysis is used to detect and quantify the presence of baldness-associated protein in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, anti-baldness-associated protein antibodies specifically bind to the baldness-associated protein on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:34-41).

4. Reduction of Non-Specific Binding

One of skill in the art will appreciate that it is often desirable to use non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this involves coating the substrate with a proteinaceous composition. In particular, protein compositions, such as bovine serum albumin (BSA), nonfat powdered milk and gelatin, are widely used.

5. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by, e.g., spectroscopic, photochemical, biochemical, immunochemical, electrical, optical or chemical means. Useful labels in the present invention include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Thyroxine and cortisol can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include, e.g., fluorescein and its derivatives, rhodamine and its derivatives, dansyl, umbelliferone, etc. Chemiluminescent compounds include, e.g., luciferin, and 2,3-dihydrophthalazinediones, e.g., luminol (for a review of various labeling or signal producing systems which may be used, see, U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

V. SCREENING FOR MODULATORS OF BALDNESS AND/OR OF HAIR-LOSS

The invention also provides methods for identifying compounds that modulate baldness and hair-loss, e.g., hair thinning, hair shortening, receding hairline, loss of hair at the crown of the head, hair-loss in a typical “M-shaped” pattern which eventually results in a loss of hair over the top of the head, complete hair loss, etc. For example, the methods can identify compounds that increase or decrease the expression level of genes and/or the activity of proteins associated with baldness and/or baldness-related conditions (e.g., hormonal imbalance, stress, thyroid disease, vitamin deficiency and/or other dietary imbalances). Although the following discussion is directed to methods for screening for modulators of baldness, similar methods can be used to screen for modulators of, e.g., hair loss, activity of hair follicles, miniaturization of hair follicles, hair growth, thinning of hair, length and thickness of hairs, etc.

For instance, compounds that are identified as modulators of baldness using the methods of the invention find use both in vitro and in vivo. For example, one can treat cell cultures with the modulators in experiments designed to determine the mechanisms by which the activity, size and/or proliferation rate of hair follicle or scalp cells is regulated. In vivo uses of compounds that delay cell hair loss include, for example, delaying baldness and/or reversing baldness and the hair loss process, as well as promoting hair growth and/or thickening.

The methods typically involve culturing a cell in the presence of a potential modulator to form a first cell culture. RNA (or cDNA) from the first cell culture is contacted with a probe which comprises a polynucleotide sequence associated with baldness. The amount of the probe which hybridizes to the RNA (or cDNA) from the first cell culture is determined. Typically, one determines whether the amount of probe which hybridizes to the RNA (or cDNA) is increased or decreased relative to the amount of the probe which hybridizes to RNA (or cDNA) from a second cell culture grown in the absence of the modulator.

It may be further determined whether the modulator-induced increase or decrease in RNA (or cDNA) levels of the target sequence is correlated with any baldness-associated change in cellular phenotype. For example, a cell population (e.g., a hair follicle cell population or a scalp cell population) that is treated with a modulator which induces decreased expression of a gene that is normally upregulated with baldness or a cell that is treated with a modulator which induces increased expression of a gene that is normally downregulated with baldness may be further tested for, e.g., regained activity, increased size, increased proliferation rate, etc.

Essentially any chemical compound can be used as a potential modulator in the assays of the invention, although most often compounds that can be dissolved in aqueous or organic (for example, DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial library containing a large number of potential therapeutic compounds (potential modulator compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991); and Houghton et al., Nature 354:84-88 (1991)). Other chemistries for generating chemical diversity libraries can also be used. Such chemistries include, but are not limited to, peptoids (WO 91/19735), encoded peptides (WO 93/20242), random bio-oligomers (WO 92/00091), benzodiazepines (U.S. Pat. No. 5,288,514), diversomers such as hydantoins, benzodiazepines and dipeptides (Hobbs et al, Proc. Nat. Acad. Sci. USA 90:6909-6913 (1993)), vinylogous polypeptides (Hagihara et al, J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel et al. Current Protocols in Molecular Biology (1987); Berger et al., supra; and Sambrook et al., supra), peptide nucleic acid libraries (see, e.g., U.S. Pat. No. 5,539,083), antibody libraries (see, e.g., Vaughn et al., Nature Biotechnology, 14(3):309-314 (1996); and PCT/US96/10287), carbohydrate libraries (see, e.g., Liang et al., Science, 274:1520-1522 (1996) and U.S. Patent 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

As noted, the invention provides in vitro assays for identifying, in a high throughput format, compounds that can modulate baldness and/or hair loss. Control reactions that measure the level of a baldness-associated protein in a cell in a reaction that does not include a potential modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions, which do not include a modulator, provide a background level of binding activity.

In some assays it will be desirable to have positive controls to ensure that the components of the assays are working properly. At least two types of positive controls are appropriate. First, a known activator of hair loss and/or baldness development can be incubated with one sample of the assay, and the resulting increase in signal resulting from an increased expression level of a gene associated with baldness determined according to the methods herein. Second, a known inhibitor of hair loss and/or baldness can be added, and the resulting decrease in signal for the expression of a gene associated with baldness similarly detected. It will be appreciated that modulators can also be combined with activators or inhibitors to find modulators which inhibit the increase or decrease that is otherwise caused by the presence of the known modulator of the development of hair loss and/or baldness.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100 to about 1500 different compounds. It is possible to assay many different plates per day; assay screens for up to about 6,000-20,000, and even up to about 100,000 different compounds are possible using the integrated systems of the invention.

VI. COMPOSITIONS, KITS AND INTEGRATED SYSTEMS

The invention provides compositions, kits and integrated systems for practicing the assays described herein. Although the following discussion is directed to kits for carrying out assays using nucleic acids (or proteins, antibodies, etc.) associated with baldness, similar kits can be assembled for carrying out assays using nucleic acids (or proteins, antibodies, etc.) associated with, e.g., hair loss, loss of activity and/or miniaturization of hair follicles, slowing of hair growth, thinning of hair, receding hairline, appearance of shorter and/or weaker hairs, etc. For instance, an assay composition having a nucleic acid associated with, for example, baldness and a labeling reagent is provided by the present invention. In some embodiments, a plurality of, for example, baldness-associated nucleic acids are provided in the assay compositions. The invention also provides assay compositions for use in solid phase assays; such compositions can include, for example, one or more baldness-associated nucleic acids immobilized on a solid support and a labeling reagent. In each case, the assay compositions can also include additional reagents that are desirable for hybridization. Modulators of expression of, for example, baldness-associated nucleic acids can also be included in the assay compositions.

The invention also provides kits for carrying out the assays of the invention. The kits typically include a probe which comprises a polynucleotide sequence associated with baldness and a label for detecting the presence of the probe. Preferably, the kits will include a plurality of polynucleotide sequences associated with baldness. Kits can include any of the compositions noted above, and optionally further include additional components such as instructions to practice a high-throughput method of assaying for an effect on expression of baldness-associated genes, one or more containers or compartments (e.g., to hold the probe, labels, or the like), a control modulator of the baldness process, a robotic armature for mixing kit components or the like.

The invention also provides integrated systems for high-throughput screening of potential modulators for an effect on the development of baldness. The systems typically include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous STAT binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT® or WINDOWS95® based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

VII. GENE THERAPY APPLICATIONS

A variety of human diseases can be treated by therapeutic approaches that involve stably introducing a gene into a human cell such that the gene is transcribed and the gene product is produced in the cell. Diseases and conditions amenable to treatment by this approach include, but are not limited to, inherited diseases, including those in which the defect is in a single gene. Gene therapy is also useful for treatment of acquired diseases and other conditions. For discussions on the application of gene therapy towards the treatment of genetic as well as acquired diseases, see, Miller Nature 357:455-460 (1992); and Mulligan Science 260:926-932 (1993).

A. Vectors for Gene Delivery

For delivery to a cell or organism, the nucleic acids of the invention can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the nucleic acids can be operably linked to expression and control sequences that can direct expression of the gene in the desired target host cells. Thus, one can achieve expression of the nucleic acid under appropriate conditions in the target cell.

B. Gene Delivery Systems

Viral vector systems useful in the expression of the nucleic acids include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including, but not limited to, Rous sarcoma virus), and MoMLV. Typically, the genes of interest are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors are also useful for introducing the nucleic acids of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins, the pol gene encodes the RNA directed DNA polymerase (reverse transcriptase), and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and the International Publication No. WO 92/07943 entitled “Retroviral Vectors Useful in Gene Therapy”.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, the baldness-associated protein and thus restore the hair follicle and/or the scalp cells to a normal active phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include, but are not limited to, Crip, GPE86, PA317 and PG13 (see Miller et al., J. Virol. 65:2220-2224 (1991)). Examples of other packaging cell lines are described in, e.g., Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

In some embodiments of the invention, an antisense nucleic acid is administered which hybridizes to a gene associated with baldness or to a transcript thereof. The antisense nucleic acid can be provided as an antisense oligonucleotide (see, e.g., Murayama et al., Antisense Nucleic Acid Drug Dev. 7:109-114 (1997)). Genes encoding an antisense nucleic acid can also be provided; such genes can be introduced into cells by methods known to those of skill in the art. For example, one can introduce a gene that encodes an antisense nucleic acid in a viral vector, such as, for example, in hepatitis B virus (see, e.g., Ji et al., J. Viral Hepat. 4:167-173 (1997)), in adeno-associated virus (see, e.g., Xiao et al., Brain Res. 756:76-83 (1997)), or in other systems including, but not limited, to an HVJ (Sendai virus)-liposome gene delivery system (see, e.g., Kaneda et al., Ann. NY Acad. Sci. 811:299-308 (1997)), a “peptide vector” (see, e.g., Vidal et al., CR Acad. Sci III 32:279-287 (1997)), as a gene in an episomal or plasmid vector (see, e.g., Cooper et al., Proc. Natl. Acad. Sci. U.S.A. 94:6450-6455 (1997), Yew et al. Hum Gene Ther. 8:575-584 (1997)), as a gene in a peptide-DNA aggregate (see, e.g., Niidome et al., J. Biol. Chem. 272:15307-15312 (1997)), as “naked DNA” (see, e.g., U.S. Pat. Nos. 5,580,859 and 5,589,466), in lipidic vector systems (see, e.g., Lee et al., Crit Rev Ther Drug Carrier Syst. 14:173-206 (1997)), polymer coated liposomes (U.S. Pat. Nos. 5,213,804 and 5,013,556), cationic liposomes (Epand et al., U.S. Pat. Nos. 5,283,185; 5,578,475; 5,279,833; and 5,334,761), gas filled microspheres (U.S. Pat. No. 5,542,935), ligand-targeted encapsulated macromolecules (U.S. Pat. Nos. 5,108,921; 5,521,291; 5,554,386; and 5,166,320).

C. Pharmaceutical Formulations

When used for pharmaceutical purposes, the vectors used for gene therapy are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. Physiologically acceptable compounds include, but are not limited to, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

D. Administration of Formulations

The formulations of the invention can be delivered to any tissue or organ using any delivery method known to the ordinarily skilled artisan. In some embodiments of the invention, the nucleic acids of the invention are formulated in topical and/or topical gel formulations. Exemplary permeation enhancing compositions, polymer matrices, and gel preparations for transdermal delivery are disclosed in, e.g., U.S. Pat. No. 5,346,701.

E. Methods of Treatment

The gene therapy formulations of the invention are typically administered to a cell. The cell can be provided as part of a tissue, such as skin, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Arteaga et al., Cancer Research 56(5):1098-1103 (1996); Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 1 1(2):416-22 (1996); and Makarov et al., Proc. Natl. Acad. Sci. USA 93(1):402-6 (1996).

VIII. ADMINISTRATION OF THE MODULATORS OF THE INVENTION AND PHARMACEUTICAL COMPOSITIONS

Modulators of the baldness-associated molecules of the present invention can be administered directly to a subject for slowing or stopping the development of baldness or for reversing baldness in vivo. Administration is by any of the routes normally used for introducing a modulator compound into ultimate contact with the tissue to be treated and well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, intravenously, or topically. The formulations of compounds can be presented in unit-dose or multi-dose sealed containers, such as ampoules and vials. Solutions and suspensions can be prepared from sterile powders, granules, and tablets of the kind previously described. The modulators can also be administered as part a of prepared food or drug. In some embodiments, the modulators are administered topically and are formulated as a cosmetic composition.

The dose administered to a patient, in the context of the present invention should be sufficient to effect a beneficial response in the subject over time. The dose will be determined by the efficacy of the particular modulators employed and the condition of the subject, as well as the body weight or surface area of the area to be treated. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. Administration can be accomplished via single or divided doses.

IX. GENERAL RECOMBINANT NUCLEIC ACIDS METHODS FOR USE WITH THE INVENTION

In numerous embodiments of the present invention, nucleic acids encoding the baldness-associated molecules of interest will be isolated and cloned using recombinant methods. Such embodiments are used, e.g., to isolate baldness-associated polynucleotides for protein expression, to monitor baldness-associated gene expression, for the isolation or detection of baldness-associated sequences in different species, for predicting the propensity for baldness in a subject, etc.

A. General Recombinant Nucleic Acids Methods

Nucleotide sizes are given in either kilobases (kb) or base pairs (bp). These are estimates derived from agarose or acrylamide gel electrophoresis or, alternatively, from published DNA sequences.

Oligonucleotides that are not commercially available can be chemically synthesized according to the solid phase phosphoramidite triester method first described by Beaucage and Caruthers, Tetrahedron Letts., 22(20):1859-1862 (1981), using an automated synthesizer, as described in Needham Van Devanter et al., Nucleic Acids Res., 12:6159-6168 (1984). Purification of oligonucleotides is, for example, by either native acrylamide gel electrophoresis or by anion-exchange HPLC as described in Pearson and Reanier, J. Chrom., 255:137-149 (1983).

The nucleic acids described here, or fragments thereof, can be used as a hybridization probe for genomic, mRNA or cDNA libraries to isolate the corresponding complete gene (including regulatory and promoter regions, exons and introns) or cDNAs, in particular cDNA clones corresponding to full length transcripts. The probes may also be used to isolate other genes and cDNAs which have a high sequence similarity to the gene of interest or similar biological activity. Probes of this type preferably have at least 30 bases and may contain, for example, 50 or more bases. Probes may also be synthetic oligonucleotides having a sequence complementary to that of a nucleic acid of interest of the present invention.

The sequence of the cloned genes and synthetic oligonucleotides can be verified using the chemical degradation method of Maxam and Gilbert Methods in Enzymology, 65:499-560 (1980). The sequence can be confirmed after the assembly of the oligonucleotide fragments into the double-stranded DNA sequence using the method of Maxam and Gilbert, supra, or the chain termination method for sequencing double-stranded templates of Wallace et al., Gene, 16:21-26 (1981). Southern blot hybridization techniques can be carried out according to Southern et al., J. Mol. Biol., 98:503 (1975).

B. Cloning Methods for the Isolation of Nucleotide Sequences Encoding the Desired Proteins

In general, the nucleic acids encoding the subject proteins are cloned from DNA sequence libraries that are made to encode copy DNA (cDNA) or genomic DNA. The particular sequences can be located by hybridizing with an oligonucleotide probe, the sequence of which can be derived from the sequences provided herein, which provides a reference for PCR primers and defines suitable regions for isolating baldness-associated specific probes. Alternatively, where the sequence is cloned into an expression library, the expressed recombinant protein can be detected immunologically with antisera or purified antibodies made against the baldness-associated protein of interest.

Methods for making and screening cDNA libraries are well known to those of skill in the art (see, e.g., Gubler and Hoffman Gene 25:263-269 (1983); and Sambrook, supra).

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, as described in Sambrook, supra, and the recombinant phages are analyzed by plaque hybridization, as described in Benton and Davis Science, 196:180-182 (1977). Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding baldness-associated proteins in physiological samples, for nucleic acid sequencing, or for other purposes (see, U.S. Pat. Nos. 4,683,195 and 4,683,202). Genes amplified by a PCR reaction can be purified, e.g., from agarose gels, and cloned into an appropriate vector.

Appropriate primers and probes for identifying genes encoding baldness-associated proteins from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A gene involved in the onset of baldness, for example, can be cloned using intermediate vectors before transformation into mammalian cells for expression. These intermediate vectors are typically prokaryote vectors or shuttle vectors. The proteins can be expressed in either prokaryotes, using standard methods well known to those of skill in the art, or eukaryotes as described infra.

C. Expression in Eukaryotes

Standard eukaryotic transfection methods are used to produce eukaryotic cell lines, e.g., yeast, insect, or mammalian cell lines, which express large quantities of the baldness-associated proteins of interest which are then purified using standard techniques (see, e.g., Colley et al., J. Biol. Chem. 264:17619-17622, (1989); and Guide to Protein Purification, in Vol. 182 of Methods in Enzymology (Deutscher ed., 1990)).

Transformations of eukaryotic cells are performed according to standard techniques as described by Morrison J. Bact., 132:349-351 (1977), or by Clark-Curtiss and Curtiss, Methods in Enzymology, 101:347-362 R. Wu et al. (Eds) Academic Press, NY (1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see Sambrook et al., supra). It is only necessary that the particular genetic engineering procedure utilized be capable of successfully introducing at least one gene into the host cell which is capable of expressing the protein.

The particular eukaryotic expression vector used to transport the genetic information into the cell is not particularly critical. Any of the conventional vectors used for expression in eukaryotic cells may be used. Expression vectors containing regulatory elements from eukaryotic viruses are typically used. Suitable vectors for use in the present invention include, but are not limited to, SV40 vectors, vectors derived from bovine papilloma virus or from the Epstein Barr virus, baculovirus vectors, and any other vector allowing expression of proteins under the direction of the SV-40 later promoter, metallothionein promoter, murine mammary tumor virus promoter, Rous sarcoma virus promoter, polyhedrin promoter, or other promoters shown effective for expression in eukaryotic cells.

The vectors usually include selectable markers which result in gene amplification, such as, e.g., thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, asparagine synthetase and ouabain selection. Alternatively, high yield expression systems not involving gene amplification are also suitable, such as, e.g., using a baculovirus vector in insect cells, with a target protein encoding sequence under the direction of the polyhedrin promoter or other strong baculovirus promoters.

The expression vector of the present invention will typically contain both prokaryotic sequences that facilitate the cloning of the vector in bacteria as well as one or more eukaryotic transcription units that are expressed only in eukaryotic cells, such as mammalian cells. The vector may or may not comprise a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the transfected DNA integrates into the genome of the transfected cell, where the promoter directs expression of the desired gene. The expression vector is typically constructed from elements derived from different, well characterized viral or mammalian genes. For a general discussion of the expression of cloned genes in cultured mammalian cells, see, Sambrook et al., supra, Ch. 16.

The prokaryotic elements that are typically included in the mammalian expression vector include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

The expression vector contains a eukaryotic transcription unit or expression cassette that contains all the elements required for the expression of the baldness-associated protein encoding DNA in eukaryotic cells. A typical expression cassette contains a promoter operably linked to the DNA sequence encoding the baldness-associated protein and signals required for efficient polyadenylation of the transcript. The DNA sequence encoding the protein may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. In preferred embodiments, the sequences of the present invention are operably linked to a heterologous promoter, i.e., the promoter directs the transcription of a sequence of interest.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. One of skill in the art would have no difficulty in selecting enhancer elements or enhancer/promoter combinations that are suitable for the present invention (see, Enhancers and Eukaryotic Expression, Cold Spring Harbor Pres, Cold Spring Harbor, N.Y. (1983)).

In the construction of the expression cassette, the promoter is preferably positioned at about the same distance from the heterologous transcription start site as it is from the transcription start site in its natural setting. As is known in the art, however, some variation in this distance can be accommodated without loss of promoter function.

In addition to a promoter sequence, the expression cassette should also contain a transcription termination region downstream of the structural gene to provide for efficient termination. The termination region may be obtained from the same gene as the promoter sequence or may be obtained from a different gene.

If the mRNA encoded by the structural gene is to be efficiently translated, polyadenylation sequences are also commonly added to the vector construct. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned genes or to facilitate the identification of cells that carry the transfected DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The cDNA encoding the protein of the invention can be ligated to various expression vectors for use in transforming host cell cultures. The vectors typically contain gene sequences to initiate transcription and translation of the baldness-associated gene of interest. These sequences need to be compatible with the selected host cell. In addition, the vectors preferably contain a marker to provide a phenotypic trait for selection of transformed host cells such as dihydrofolate reductase or metallothionein. Additionally, a vector might contain a replicative origin.

Cells of mammalian origin are illustrative of cell cultures useful for the production of, for example, a baldness-associated protein of interest. Mammalian cell systems often will be in the form of monolayers of cells although mammalian cell suspensions may also be used. Illustrative examples of mammalian cell lines include, but are not limited to, VERO and HeLa cells, Chinese hamster ovary (CHO) cell lines, W138, BHK, COS-7 or MDCK cell lines, and NIH 3T3 and COS cells.

As indicated above, the vector, e.g., a plasmid, which is used to transform the host cell, preferably contains DNA sequences to initiate transcription and sequences to control the translation of the baldness-associated protein gene sequence. These sequences are referred to as expression control sequences. Illustrative expression control sequences are obtained from the SV-40 promoter (Berman et al. Science, 222:524-527 (1983)), the CMV I.E. Promoter (Thomsen et al. Proc. Natl. Acad. Sci. 81:659-663 (1984)) or the metallothionein promoter (Brinster et al. Nature 296:39-42 (1982)). The cloning vector containing the expression control sequences is cleaved using restriction enzymes, adjusted in size as necessary or desirable and ligated with sequences encoding the baldness-associated protein by means well known in the art.

When higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague et al., J. Virol. 45:773-781 (1983)).

Additionally, gene sequences to control replication in the host cell may be incorporated into the vector such as those found in bovine papilloma virus type-vectors (see, Saveria-Campo “Bovine Papilloma virus DNA a Eukaryotic Cloning Vector” In: DNA Cloning Vol.II: a Practical Approach (Glover Ed.), IRL Press, Arlington, Va. pp. 213-238 (1985)).

The transformed cells are cultured by means well known in the art. For example, such means are published in Biochemical Methods in Cell Culture and Virology, Kuchler, Dowden, Hutchinson and Ross, Inc. (1977). The expressed protein is isolated from cells grown as suspensions or as monolayers. The latter are recovered by well known mechanical, chemical or enzymatic means.

X. PURIFICATION OF THE PROTEINS FOR USE WITH THE INVENTION

After expression, the proteins of the present invention can be purified to substantial purity by standard techniques, including, but not limited to, selective precipitation with substances as ammonium sulfate, column chromatography, immunopurification methods, and other methods known to those of skill in the art (see, e.g., Scopes Protein Purification: Principles and Practice, Springer-Verlag, NY (1982); U.S. Pat. No. 4,673,641; Ausubel et al., supra; and Sambrook et al., supra).

A number of conventional procedures can be employed when a recombinant protein is being purified. For example, proteins having established molecular adhesion properties can be reversibly fused to the subject protein. With the appropriate ligand, a baldness-associated protein of interest, for example, can be selectively adsorbed to a purification column and then freed from the column in a relatively pure form. The fused protein is then removed by enzymatic activity. Finally, a baldness-associated protein of interest can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al., and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing reformation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques For Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

It will be apparent to one of skill that chromatographic techniques can be performed at any scale and using equipment from many different manufacturers (e.g., Pharmacia Biotech).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Table 1 below indicates genes by identification in the “LifeSpan Cluster name” column that demonstrate a change in expression with baldness in samples from male, human scalp. “LifeSpan HAD ID” indicates the clone identification number in the LifeSpan High Density Arrays collection. “LifeSpan Cluster ID” refers to the clone identification number in the LifeSpan collection of clusters. “Image CloneID” refers to the IMAGE Consortium library clone identification number.

In Table 1A, the “NonBald-Bald ratio” column indicates for a given gene the ratio of the expression of the gene in a non-bald individual or in a non-bald region of the scalp of an individual versus the expression of the gene in a bald individual or in a bald region of the scalp of an individual. A gene with a “NonBald-Bald ratio” >1 (e.g., LFP40) is a gene that is expressed at a higher level in non-bald individuals or in non-bald regions of the scalp than in bald individuals or in bald regions of the scalp, i.e., a gene that is downregulated with baldness. Conversely, a gene with a “NonBald-Bald ratio” <1 is a gene that is expressed at a lower level in non-bald individuals or in non-bald regions of the scalp than in bald individuals or in bald regions of the scalp, i.e., a gene that is upregulated with baldness.

In Table 1B, the “Bald-Transit ratio” column indicates for a given gene, the ratio of the expression of the gene in bald individuals or in bald regions of the scalp versus transitional individuals or transitional regions of the scalp. A gene with a “Bald-Transit ratio” >1 is a gene that is expressed at a higher level in a bald individual or in a bald region of the scalp than in a transitional individual or a transitional region of the scalp. Such a gene is upregulated with baldness. Conversely, a gene with a “Bald-Transit ratio” <1 is a gene that is expressed at a lower level in a bald individual or in a bald region of the scalp than in a transitional individual or a transitional region of the scalp, and is, thus, a gene that is downregulated with baldness.

Finally, in Table 1 C, the “NonBald-TransitPhase ratio” column shows the ratio of the expression of a given gene in non-bald individuals or in non-bald regions of the scalp versus the expression of the gene in transitional individuals or in transitional regions of the scalp. A gene with a “NonBald-TransitPhase ratio” >1 is a gene that is expressed at a higher level in a non-bald individual or in a non-bald region of the scalp than in a transitional individual or a transitional region of the scalp, and is, thus, a gene that is downregulated with baldness. A gene with a “NonBald-TransitPhase ratio” <1 is a gene that is expressed at a lower level in a non-bald individual or in a non-bald region of the scalp than in a transitional individual or a transitional region of the scalp, and is, thus, a gene that is upregulated with baldness.

TABLE 1A

| LifeSpan | LifeSpan | | Image | NonBald |
|---|---|---|---|---|
| HDA ID | ClusterID | LifeSpan Cluster name | CloneID | Bald ratio |
| 2859 | 5875 | IMMUNOGLOBULIN LIGHT CHAIN | 162999 | 35.93 |
| 598 | 16147 | GUANINE NUCLEOTIDE REGULATORY FACTOR (LFP40) | 25328 | 12.9 |
| 163 | 1846 | GLUCOSE-6-PHOSPHATE 1-DEHYDROGENASE | 32644 | 12 |
| 6950 | 3655 | PEPTIDYL-PROLYL CIS-TRANS ISOMERASE, | 49281 | 9.336 |
| | | MITOCHONDRIAL | | |
| 3033 | 138644 | 1 RECORD MOVED FROM CLUSTER 56351 | 184256 | 8.911 |
| 7211 | 822 | CAMP-DEPENDENT PROTEIN KINASE TYPE II-BETA | 310390 | 7.619 |
| | | REGULATORY CHAIN | | |
| 2673 | 56267 | ANTIGEN NY-CO-31 (NY-CO-31) | 152748 | 7.525 |
| 3394 | 4218 | PHOSPHODIESTERASE PDE6G | 219980 | 6.595 |
| 6728 | 3917 | PROPIONYL-COA CARBOXYLASE ALPHA CHAIN | 109986 | 5.822 |
| 6287 | 492 | APOLIPOPROTEIN E RECEPTOR 2 | 649804 | 5.703 |
| 764 | 23011 | ESTS, HIGHLY SIMILAR TO NADH-CYTOCHROME B5 | 31831 | 5.647 |
| | | REDUCTASE [BOS TAURUS] | | |
| 2396 | 1880 | GLUTATHIONE S-TRANSFERASE MU 3 | 137940 | 4.933 |
| 5202 | 2815 | KI NUCLEAR AUTOANTIGEN | 486060 | 4.638 |
| 2517 | 3891 | COLLAGEN ALPHA1 (I) | 143925 | 4.551 |
| 6104 | 4707 | THYMOSIN BETA-4 | 594922 | 4.52 |
| 6441 | 4917 | TUBULIN BETA-1 CHAIN | 727352 | 4.313 |
| 5181 | 1664 | FIBULIN-2 | 485648 | 4.133 |
| 7106 | 39922 | ESTS | 264336 | 4.103 |
| 6559 | 2772 | ISOVALERYL-COA DEHYDROGENASE | 756073 | 4.062 |
| 986 | 33121 | HEVIN LIKE PROTEIN | 41629 | 3.742 |
| 1268 | 4625 | T-CELL RECEPTOR GAMMA CHAIN | 66322 | 3.712 |
| 5225 | 4329 | SEMAPHORIN E | 486591 | 3.672 |
| 2402 | 4831 | TRANSCRIPTIONAL REGULATOR ISGF3 GAMMA SUBUNIT | 138181 | 3.663 |
| 5166 | 3585 | P-SELECTIN GLYCOPROTEIN LIGAND 1 | 484838 | 3.543 |
| 3743 | 48224 | MUF1 PROTEIN | 258835 | 3.543 |
| 4144 | 119463 | 1 RECORD MOVED FROM CLUSTER 18902 | 290420 | 3.522 |
| 2212 | 1497 | ENOYL-COA HYDRATASE, MITOCHONDRIAL | 128447 | 3.449 |
| 622 | 2915 | LEUKOCYTE ANTIGEN CD37 | 26202 | 3.316 |
| 5154 | 2804 | KERATIN 6 ISOFORM K6E (KRT6E) | 472084 | 3.304 |
| 5591 | 2891 | LAMININ BETA-3 CHAIN | 526215 | 3.229 |
| 721 | 3152 | MICROTUBULE-ASSOCIATED PROTEIN 1B | 29706 | 3.107 |
| 7090 | 5525 | GTPASE-ACTIVATING PROTEIN (SIPA1) | 259060 | 3.061 |
| 6802 | 150811 | 2 RECORDS MOVED FROM CLUSTER 148964 | 35084 | 3 |
| 1044 | 18639 | ESTS | 37839 | 3 |
| 957 | 4861 | TRANSLATION INITIATION FACTOR EIF-2B EPSILON | 40360 | 2.993 |
| | | SUBUNIT | | |
| 423 | 3733 | PITUITARY HOMEOBOX 1 | 627344 | 2.99 |
| 246 | 1194 | CYTOCHROME P450 IVF3 | 78317 | 2.934 |
| 2475 | 638 | BETA-1,4-GALACTOSYLTRANSFERASE | 141570 | 2.927 |
| 6190 | 3429 | NF-AT4C | 625934 | 2.923 |
| 4703 | 139828 | 4 RECORDS MOVED FROM CLUSTER 77323 | 360595 | 2.911 |
| 1309 | 3287 | MYOSIN LIGHT CHAIN ALKALI, SMOOTH-MUSCLE ISOFORM | 68163 | 2.882 |
| 2836 | 178848 | 1 RECORD MOVED FROM CLUSTER 825 | 160729 | 2.88 |
| 6826 | 5391 | A-KINASE ANCHOR PROTEIN (AKAP100) | 40844 | 2.805 |
| 4544 | 27066 | ESTS, HIGHLY SIMILAR TO COATOMER ZETA SUBUNIT | 323753 | 2.8 |
| | | [BOS TAURUS] | | |
| 545 | 4661 | TENASCIN | 23532 | 2.798 |
| 2999 | 56337 | E. COLI GENOMIC DNA, KOHARA CLONE #272 (32.4-32.7 MIN.) | 179902 | 2.793 |
| 3516 | 4296 | S100 CALCIUM-BINDING PROTEIN A2 | 238479 | 2.787 |
| 5924 | 57063 | ESCHERICHIA COLI GENOMIC DNA. (16.1-16.4 MIN) | 563439 | 2.735 |
| 1538 | 120194 | 3 RECORDS MOVED FROM CLUSTER 19417 | 78262 | 2.721 |
| 5729 | 154268 | 59 RECORDS MOVED FROM CLUSTER 917 | 544806 | 2.675 |
| 7095 | 44656 | ESTS | 261519 | 2.671 |
| 5075 | 7117 | 39 KDA PROTEIN | 469977 | 2.641 |
| 1850 | 843 | CARBONYL REDUCTASE | 113023 | 2.546 |
| 6080 | 155858 | 1 RECORD MOVED FROM CLUSTER 750 | 593166 | 2.509 |
| 5654 | 139136 | 1 RECORD MOVED FROM CLUSTER 56932 | 530375 | 2.506 |
| 5150 | 154265 | ERBA-RELATED PROTEIN EAR-3 | 471889 | 2.484 |
| 5441 | 40887 | ESTS, MODERATELY SIMILAR TO MITOCHONDRIAL | 504351 | 2.455 |
| | | CARNITINE PALMITOYLTRANSFERASE I [RATTUS | | |
| | | NORVEGICUS] | | |
| 5508 | 100993 | 1 RECORD MOVED FROM CLUSTER 5160 | 510412 | 2.418 |
| 5507 | 2352 | KIAA0043 | 510388 | 2.404 |
| 1232 | 18656 | ESTS, HIGHLY SIMILAR TO CMP-N-ACETYLNEURAMINATE- | 51210 | 2.391 |
| | | BETA-1,4-GALACTOSIDE ALPHA-2,3-SIALYLTRANSFERASE | | |
| | | [RATTUS NORVEGICUS] | | |
| 2887 | 4693 | THROMBOSPONDIN 3 | 166893 | 2.378 |
| 7621 | 566 | ATP-DEPENDENT RNA HELICASE A | 30626 | 2.368 |
| 5239 | 1924 | GONADOLIBERIN | 487071 | 2.347 |
| 6508 | 3780 | PLECTIN | 741727 | 2.336 |
| 3391 | 138741 | 1 RECORD MOVED FROM CLUSTER 56455 | 219851 | 2.304 |
| 475 | 20258 | ESTS, MODERATELY SIMILAR TO DIAMINE | 21667 | 2.301 |
| | | ACETYLTRANSFERASE [MUS MUSCULUS] | | |
| 1448 | 7350 | NADH-UBIQUINONE OXIDOREDUCTASE SUBUNIT CI-B12 | 73817 | 2.298 |
| 177 | 4621 | T-CELL SURFACE GLYCOPROTEIN CD1D | 47266 | 2.281 |
| 1271 | 121522 | 1 RECORD MOVED FROM CLUSTER 20340 | 66378 | 2.264 |
| 7113 | 5255 | ZINC FINGER PROTEIN 40 | 266854 | 2.252 |
| 7419 | 18149 | ESTS | 502446 | 2.233 |
| 2438 | 3401 | NEURONATIN | 139681 | 2.218 |
| 7240 | 4844 | TRANSFORMING GROWTH FACTOR BETA 2 | 326155 | 2.214 |
| 5990 | 1623 | FATTY ACID-BINDING PROTEIN, ADIPOCYTE | 567306 | 2.207 |
| 6910 | 56102 | ALPHA-FETOPROTEIN ENHANCER BINDING PROTEIN | 158983 | 2.197 |
| 6101 | 24540 | ESTS, HIGHLY SIMILAR TO PROBABLE 26S PROTEASE | 594796 | 2.186 |
| | | SUBUNIT YTA6 [SACCHAROMYCES CEREVISIAE] | | |
| 3873 | 3458 | NONSPECIFIC LIPID-TRANSFER PROTEIN; sterol carrier protein | 267357 | 2.176 |
| | | X/sterol carrier protein 2 | | |
| 1034 | 14217 | TUMOR NECROSIS FACTOR TYPE 2 RECEPTOR ASSOCIATED | 42716 | 2.169 |
| | | PROTEIN (TRAP3) | | |
| 6365 | 2967 | LYMPHOCYTE ANTIGEN 64 | 700650 | 2.166 |
| 2047 | 19346 | ESTS, HIGHLY SIMILAR TO COMPLEMENT RECEPTOR TYPE | 121678 | 2.157 |
| | | 2 PRECURSOR [MUS MUSCULUS] | | |
| 7222 | 38088 | ESTS | 323396 | 2.13 |
| 5625 | 172 | RIBOSOMAL PROTEIN L19 | 529388 | 2.126 |
| 2715 | 86640 | 29 RECORDS MOVED FROM CLUSTER 1341 | 154053 | 2.106 |
| 7276 | 27551 | ESTS | 343437 | 2.104 |
| 5379 | 1870 | GLUTAREDOXIN | 501952 | 2.104 |
| 5260 | 2794 | KERATIN, TYPE I CYTOSKELETAL 18 | 487868 | 2.103 |
| 3007 | 14595 | ARGBPIB PROTEIN | 180813 | 2.102 |
| 2034 | 14 | 1,4-ALPHA-GLUCAN BRANCHING ENZYME | 121163 | 2.101 |
| 7386 | 24515 | ESTS | 485941 | 2.092 |
| 1193 | 8977 | KIAA0241 | 49139 | 2.077 |
| 3510 | 132054 | 2 RECORDS MOVED FROM CLUSTER 30139 | 236210 | 2.071 |
| 3513 | 135825 | 1 RECORD MOVED FROM CLUSTER 44497 | 238349 | 2.068 |
| 1824 | 2372 | HYDROXYMETHYLGLUTARYL-COA SYNTHASE, | 111974 | 2.067 |
| | | CYTOPLASMIC | | |
| 760 | 3580 | OX40L RECEPTOR | 32008 | 2.063 |
| 4504 | | | 322429 | 2.061 |
| 5102 | 2799 | KERATIN, TYPE II CYTOSKELETAL 2 EPIDERMAL | 470517 | 2.058 |
| 4941 | 1280 | DESMOCOLLIN 2A/2B | 427989 | 2.051 |
| 2056 | 3831 | PREGNANCY SPECIFIC BETA-1-GLYCOPROTEIN 4 (PSG4) | 122004 | 2.026 |
| 3441 | 4338 | SERINE-PYRUVATE AMINOTRANSFERASE | 229650 | 2.019 |
| 2611 | 15415 | INOSITOL POLYPHOSPHATE 5-PHOSPHATASE | 149771 | 2.014 |
| 4724 | 2838 | KIAA0146 PROTEIN | 361107 | 0.499 |
| 1548 | 748 | C-REACTIVE PROTEIN | 78639 | 0.499 |
| 3136 | 23837 | ESTS, HIGHLY SIMILAR TO DEOXYRIBOSE-PHOSPHATE | 195384 | 0.498 |
| | | ALDOLASE [ESCHERICHIA COLI] | | |
| 3918 | 11020 | ENDOGENOUS RETROVIRUS TYPE C | 270385 | 0.498 |
| 2372 | 39965 | ESTS, WEAKLY SIMILAR TO NADH-UBIQUINONE | 136708 | 0.498 |
| | | OXIDOREDUCTASE CHAIN 5 [PARAMECIUM TETRAURELIA] | | |
| 2250 | 183984 | 1 RECORD MOVED FROM CLUSTER 16848 | 130216 | 0.498 |
| 3289 | 1971 | GTP: AMP PHOSPHOTRANSFERASE MITOCHONDRIAL | 207155 | 0.497 |
| 4479 | 2339 | HOMEOBOX PROTEIN MSX-2 | 322016 | 0.497 |
| 5667 | 125 | 5-AMINOLEVULINIC ACID SYNTHASE MITOCHONDRIAL | 530775 | 0.495 |
| | | PRECURSOR, NONSPECIFIC | | |
| 5042 | 2846 | KIAA0221 | 469220 | 0.495 |
| 6091 | 6168 | SMAD5 | 594181 | 0.494 |
| 1840 | 117640 | 59 RECORDS MOVED FROM CLUSTER 17389 | 112506 | 0.493 |
| 6610 | 6632 | HOMEOBOX PROTEIN SHOTB | 773219 | 0.491 |
| 5818 | 57038 | | 547068 | 0.489 |
| 2932 | 1322 | DIPHOSPHOMEVALONATE DECARBOXYLASE | 173661 | 0.489 |
| 4769 | 5145 | VOLTAGE-GATED POTASSIUM CHANNEL PROTEIN KV1.1 | 362585 | 0.488 |
| 4434 | 2740 | INTERLEUKIN-6 | 310406 | 0.488 |
| 6335 | 2960 | LUMICAN | 682425 | 0.488 |
| 583 | 2391 | HYPOTHETICAL PROTEIN KIAA0056 | 25062 | 0.488 |
| 2728 | 2785 | KERATIN, TYPE II HAIR-SPECIFIC | 154446 | 0.487 |
| 6619 | 181314 | 1 RECORD MOVED FROM CLUSTER 3239 | 773422 | 0.486 |
| 2148 | 494 | APOLIPOPROTEIN (A) | 125725 | 0.485 |
| 4408 | 4739 | TISSUE ALPHA-L-FUCOSIDASE | 308437 | 0.485 |
| 6456 | 179428 | 40 RECORDS MOVED FROM CLUSTER 1155 | 728041 | 0.485 |
| 2231 | 8671 | L-KYNURENINE HYDROLASE | 129161 | 0.485 |
| 6984 | 56309 | | 173389 | 0.483 |
| 4742 | 114198 | 76 RECORDS MOVED FROM CLUSTER 14857 | 362023 | 0.481 |
| 6490 | 8439 | KIAA0061 | 731728 | 0.478 |
| 6852 | 39282 | ESTS | 132515 | 0.478 |
| 5751 | 57005 | DIAZEPAM-BINDING INHIBITOR | 545081 | 0.475 |
| 6980 | 39717 | ESTS | 172418 | 0.472 |
| 5925 | 5508 | PROTEIN KINASE PKU-ALPHA | 563451 | 0.472 |
| 540 | 56484 | | 23266 | 0.472 |
| 7591 | 718 | BRCA2 | 711698 | 0.469 |
| 2426 | 4881 | TRICHOHYALIN | 139143 | 0.468 |
| 5929 | 3169 | MITOCHONDRIAL LON PROTEASE HOMOLOG | 563611 | 0.467 |
| 1704 | 486 | APOLIPOPROTEIN C-I | 85916 | 0.466 |
| 286 | 19096 | ESTS, WEAKLY SIMILAR TO DIAPHANOUS PROTEIN | 298048 | 0.465 |
| | | [D. MELANOGASTER] | | |
| 7648 | 1615 | FANCONI ANEMIA GROUP C PROTEIN | 236355 | 0.465 |
| 4075 | 48859 | ESTS | 282295 | 0.465 |
| 1370 | 308 | ADRENAL SPECIFIC 30 KD PROTEIN | 70777 | 0.464 |
| 5437 | 2774 | ITBA2 PROTEIN | 504292 | 0.464 |
| 845 | 13531 | ALPHA1-SYNTROPHIN (SNT A1) | 35230 | 0.463 |
| 7010 | 39721 | ESTS | 187591 | 0.463 |
| 5595 | 139125 | 1 RECORD MOVED FROM CLUSTER 56897 | 526280 | 0.462 |
| 7375 | 52868 | EST | 469638 | 0.46 |
| 3283 | 5306 | ZONA PELLUCIDA SPERM-BINDING PROTEIN 3A | 206719 | 0.459 |
| 5436 | 6964 | CLONE 23956 | 504289 | 0.458 |
| 1036 | 790 | CALCYPHOSINE | 42992 | 0.458 |
| 5718 | 3091 | MESOTHELIN | 544548 | 0.458 |
| 7406 | 134667 | 37 RECORDS MOVED FROM CLUSTER 39938 | 489983 | 0.457 |
| 7163 | 129287 | 1 RECORD MOVED FROM CLUSTER 26618 | 288919 | 0.456 |
| 6691 | 59858 | | 75268 | 0.456 |
| 623 | 4265 | RIBOSE-PHOSPHATE PYROPHOSPHOKINASE III | 26210 | 0.454 |
| 6970 | 25914 | ESTS | 171557 | 0.454 |
| 501 | 683 | BLEOMYCIN HYDROLASE | 22483 | 0.454 |
| 6170 | 3430 | NF-ATC TRANSCRIPTION FACTOR | 613074 | 0.453 |
| 1233 | 6352 | THIOESTERASE II | 50898 | 0.451 |
| 3970 | 129480 | 28 RECORDS MOVED FROM CLUSTER 26822 | 274405 | 0.451 |
| 6862 | 21217 | ESTS | 135634 | 0.45 |
| 1558 | 40103 | ESTS, MODERATELY SIMILAR TO ANTIFREEZE | 79146 | 0.445 |
| | | GLYCOPEPTIDE POLYPROTEIN AFGP7/AFGP8 PRECURSOR | | |
| | | [NOTOTHENIA CORIICEPS NEGLECTA] | | |
| 6572 | 26619 | ESTS | 758420 | 0.445 |
| 4317 | 2083 | INHIBIN BETA C CHAIN | 301507 | 0.445 |
| 717 | 422 | AMILORIDE-SENSITIVE SODIUM CHANNEL BNAC1 | 29692 | 0.444 |
| 3106 | 18886 | ESTS | 193900 | 0.444 |
| 5790 | 1592 | EXCITATORY AMINO ACID TRANSPORTER 3 | 546398 | 0.444 |
| 6428 | 1944 | GRB14 | 726559 | 0.441 |
| 4172 | 138901 | 1 RECORD MOVED FROM CLUSTER 56637 | 292434 | 0.441 |
| 2102 | 2115 | HIGH MOBILITY GROUP PROTEIN HMG2 | 124257 | 0.438 |
| 1338 | 13844 | EXTRACELLULAR PROTEIN (S1-5) | 69280 | 0.436 |
| 2688 | 5530 | AQUAPORIN-7 LIKE | 153310 | 0.431 |
| 476 | 20231 | ESTS, HIGHLY SIMILAR TO PROTEIN PHOSPHATASES PP1 | 21471 | 0.431 |
| | | REGULATORY SUBUNIT SDS22 [SCHIZOSACCHAROMYCES | | |
| | | POMBE] | | |
| 1459 | 1672 | FLAVIN REDUCTASE | 74162 | 0.43 |
| 5157 | 3246 | MYELOBLAST KIAA0223 | 484504 | 0.429 |
| 4603 | 4856 | TRANSITIONAL ENDOPLASMIC RETICULUM ATPASE | 328401 | 0.428 |
| 6975 | 25981 | ESTS | 172140 | 0.427 |
| 7470 | 57018 | | 545704 | 0.425 |
| 301 | 401 | ALPHA-L-IDURONIDASE | 325954 | 0.424 |
| 5879 | 1894 | GLYCINE CLEAVAGE SYSTEM H PROTEIN | 549934 | 0.42 |
| 6798 | 125608 | 4 RECORDS MOVED FROM CLUSTER 23238 | 33022 | 0.42 |
| 6754 | 137397 | 1 RECORD MOVED FROM CLUSTER 48960 | 120513 | 0.419 |
| 6996 | 25976 | ESTS | 178488 | 0.417 |
| 5796 | 21239 | ESTS, HIGHLY SIMILAR TO HYPOTHETICAL 38.2 KD | 546573 | 0.416 |
| | | PROTEIN IN BEM2-SPT2 INTERGENIC REGION | | |
| | | [SACCHAROMYCES CEREVISIAE] | | |
| 5739 | 56999 | | 544952 | 0.416 |
| 5899 | 4900 | TROPONIN T, FAST SKELETAL MUSCLE ISOFORM BETA | 562249 | 0.414 |
| 6316 | 1299 | DIHYDROLIPOAMIDE DEHYDROGENASE | 666339 | 0.414 |
| 2754 | 33493 | 5T4 GENE FOR 5T4 ONCOFETAL ANTIGEN | 155195 | 0.413 |
| 4904 | 135686 | 1 RECORD MOVED FROM CLUSTER 44355 | 417434 | 0.413 |
| 7062 | 1732 | G1/S-SPECIFIC CYCLIN D3 | 240171 | 0.411 |
| 1169 | 25799 | ESTS, WEAKLY SIMILAR TO CELLULAR RETINALDEHYDE- | 47625 | 0.409 |
| | | BINDING PROTEIN [BOS TAURUS] | | |
| 1357 | 26563 | ESTS, HIGHLY SIMILAR TO MITOCHONDRIAL RNA | 70177 | 0.408 |
| | | SPLICING PROTEIN MSR4 [SACCHAROMYCES CEREVISIAE] | | |
| 2967 | 4809 | TRANSCRIPTION INITIATION FACTOR IIF, ALPHA SUBUNIT | 177140 | 0.407 |
| 5315 | 17748 | ESTS, WEAKLY SIMILAR TO HYPOTHETICAL 26.1 KD | 489051 | 0.406 |
| | | PROTEIN IN RIB5-SHM1 INTERGENIC REGION | | |
| | | [SACCHAROMYCES CEREVISIAE] | | |
| 406 | 4682 | THIOL-SPECIFIC ANTIOXIDANT | 531464 | 0.405 |
| 4370 | 14744 | NUCLEOSIDE DIPHOSPHATE KINASE HOMOLOG (DR-NM23) | 305533 | 0.405 |
| 2130 | 594 | B-LYMPHOCYTE ACTIVATION MARKER BLAST-1 | 125134 | 0.404 |
| 6420 | 610 | BASIC TRANSCRIPION FACTOR 2, 44 KD SUBUNIT | 726071 | 0.401 |
| 5044 | 3922 | PROSTACYCLIN SYNTHASE | 469275 | 0.4 |
| 2171 | 5630 | KIAA0439 | 126828 | 0.399 |
| 6150 | 57105 | TITIN Z-DISC | 611590 | 0.398 |
| 3773 | 9677 | PREGNANCY-SPECIFIC BETA-1-GLYCOPROTEIN PSG95 | 260126 | 0.397 |
| 2048 | 1638 | FIBRILLIN 2 | 121722 | 0.389 |
| 4212 | 4942 | TYPE III IODITHYRONINE DEIODINASE | 296032 | 0.388 |
| 6260 | 5071 | URIDINE DIPHOSPHOGLUCOSE PYROPHOSPHORYLASE | 646513 | 0.386 |
| 1244 | 3534 | OLIGODENDROCYTE-MYELIN GLYCOPROTEIN | 51373 | 0.383 |
| 6627 | 3921 | Prostacyclin receptor | 774146 | 0.381 |
| 971 | 6736 | NEUROCAN (CSPG3) | 41261 | 0.38 |
| 7654 | 4225 | RETINOBLASTOMA SUSCEPTIBILITY PROTEIN RB1 | 257626 | 0.379 |
| 1780 | 4860 | TRANSLATION INITIATION FACTOR EIF-2B ALPHA SUBUNIT | 110534 | 0.379 |
| 4807 | 2785 | KERATIN, TYPE II HAIR-SPECIFIC | 365043 | 0.379 |
| 1390 | 8548 | CH-TOG PROTEIN | 71657 | 0.379 |
| 6560 | 2873 | LACTASE-PHLORIZIN HYDROLASE | 756878 | 0.378 |
| 5498 | 1581 | EUKARYOTIC TRANSLATION INITIATION FACTOR 3 BETA | 510245 | 0.376 |
| | | SUBUNIT | | |
| 2979 | 3889 | PROCHOLECYSTOKININ | 178091 | 0.375 |
| 5941 | 139180 | 1 RECORD MOVED FROM CLUSTER 57065 | 564205 | 0.373 |
| 6599 | 4797 | TRANSCRIPTION FACTOR SP2 | 770397 | 0.371 |
| 869 | 2902 | LEGUMAIN | 36128 | 0.368 |
| 497 | 3276 | MYOSIN HEAVY CHAIN, NONMUSCLE TYPE A | 22140 | 0.368 |
| 4290 | 1961 | GS2 PROTEIN | 300163 | 0.367 |
| 2736 | 1374 | DNA-DIRECTED RNA POLYMERASE II 13.3 KD POLYPEPTIDE | 154544 | 0.367 |
| 1444 | 14723 | SELENIUM-BINDING PROTEIN (HSBP) | 73737 | 0.365 |
| 4899 | 14854 | SPLICING FACTOR SRP30C | 417073 | 0.365 |
| 4697 | 38171 | ESTS, WEAKLY SIMILAR TO TWITCHIN [C. ELEGANS] | 360472 | 0.364 |
| 6979 | 8335 | HETEROGENEOUS NUCLEAR RIBONUCLEOPROTEINS C1/C2 | 172356 | 0.362 |
| 7623 | 1343 | DNA POLYMERASE GAMMA | 32577 | 0.362 |
| 6171 | 16066 | CC3 (CC3) | 613246 | 0.361 |
| 2897 | 15388 | M. FASCICULARIS MRNA FOR NAD+−ISOCITRATE | 171786 | 0.357 |
| | | DEHYDROGENASE | | |
| 437 | 2973 | LYMPHOTOXIN-BETA | 711697 | 0.353 |
| 4896 | 141607 | 2 RECORDS MOVED FROM CLUSTER 123787 | 417024 | 0.35 |
| 1666 | 1483 | ENDOPLASMIN | 83465 | 0.347 |
| 6982 | 124878 | 58 RECORDS MOVED FROM CLUSTER 22719 | 172567 | 0.346 |
| 4639 | 472 | ANTIOXIDANT ENZYME AOE372 | 343353 | 0.339 |
| 6274 | 57124 | | 648411 | 0.337 |
| 1916 | 56077 | Orphan G protein-coupled receptor 56077 | 115277 | 0.335 |
| 1611 | 33367 | E-MAP-115 | 80734 | 0.332 |
| 5036 | 1837 | GLUCOCORTICOID RECEPTOR REPRESSION FACTOR 1 | 430335 | 0.326 |
| 6093 | 477 | AP-2 GAMMA TRANSCRIPTION FACTOR | 594372 | 0.325 |
| 2713 | 33116 | ESTS | 154032 | 0.324 |
| 4984 | 1977 | GUANINE NUCLEOTIDE EXCHANGE FACTOR PROTEIN TRIO | 429234 | 0.323 |
| 5340 | 56856 | ESTS, WEAKLY SIMILAR TO HYPOTHETICAL 32.0 KD | 489814 | 0.32 |
| | | PROTEIN IN SAP190-SPO14 INTERGENIC REGION | | |
| | | [SACCHAROMYCES CEREVISIAE] | | |
| 960 | 3473 | NUCLEAR HORMONE RECEPTOR NOR-1 | 40831 | 0.318 |
| 2570 | 8798 | E14 PROTEIN; NPAT | 146987 | 0.316 |
| 2964 | 1542 | ERPROT 213-21 | 176786 | 0.315 |
| 238 | 601 | B4-2 PROTEIN | 61261 | 0.312 |
| 849 | 1901 | GLYCOGEN (STARCH) SYNTHASE, MUSCLE | 35615 | 0.312 |
| 7093 | 3831 | PREGNANCY SPECIFIC BETA-1-GLYCOPROTEIN 4 (PSG4) | 259818 | 0.311 |
| 5808 | 139168 | 1 RECORD MOVED FROM CLUSTER 57032 | 546829 | 0.311 |
| 3892 | 817 | PHOSPHODIESTERASE PDE4D | 268455 | 0.305 |
| 1040 | 3024 | MALATE OXIDOREDUCTASE | 42910 | 0.302 |
| 4799 | 4158 | RAS-RELATED PROTEIN RAB-1A | 363872 | 0.301 |
| 5146 | 156057 | 1 RECORD MOVED FROM CLUSTER 855 | 471861 | 0.292 |
| 879 | 125640 | 4 RECORDS MOVED FROM CLUSTER 23267 | 36716 | 0.29 |
| 5486 | 519 | ARGININOSUCCINATE LYASE | 510018 | 0.29 |
| 260 | 1585 | EUKARYOTIC TRANSLATION INITIATION FACTOR 5 | 113597 | 0.287 |
| 4710 | 39778 | ESTS, HIGHLY SIMILAR TO SODIUM-INDEPENDENT | 360813 | 0.285 |
| | | ORGANIC ANION TRANSPORTER [RATTUS NORVEGICUS] | | |
| 5421 | 2386 | HYPOTHETICAL PROTEIN | 503809 | 0.282 |
| 6721 | 148 | 6-PHOSPHOFRUCTO-2-KINASE (LIVER ISOZYME) | 86044 | 0.281 |
| 1125 | 273 | Adenosine A2a receptor | 45788 | 0.281 |
| 5725 | 56985 | | 544693 | 0.278 |
| 7334 | 4769 | TRANSCRIPTION FACTOR E2-ALPHA | 366893 | 0.274 |
| 6977 | 25933 | ESTS, HIGHLY SIMILAR TO SYNTAXIN A [BOS TAURUS] | 172237 | 0.264 |
| 7091 | 7549 | CLONE 24684 | 259162 | 0.263 |
| 7397 | 3840 | PROBABLE ACTIN-BINDING PROTEIN ACF7 | 488364 | 0.262 |
| 7136 | 5619 | MBNL PROTEIN | 271915 | 0.255 |
| 1682 | 120918 | 34 RECORDS MOVED FROM CLUSTER 20034 | 84191 | 0.252 |
| 3232 | 56404 | T-STAR (T-STAR) | 202836 | 0.252 |
| 1126 | 4593 | SYNTAXIN 3 | 45789 | 0.251 |
| 604 | 22592 | ESTS, HIGHLY SIMILAR TO FIBROPELLIN C PRECURSOR | 25810 | 0.25 |
| | | [STRONGYLOCENTROTUS PURPURATUS] | | |
| 7399 | 31750 | ESTS | 488683 | 0.247 |
| 7444 | 5005 | UBIQUITIN-CONJUGATING ENZYME E2-18 KD | 511757 | 0.244 |
| 2693 | 16820 | NOVEL T-CELL ACTIVATION PROTEIN | 153460 | 0.241 |
| 3008 | 15235 | CDC37 HOMOLOG | 181066 | 0.24 |
| 3662 | 30158 | RY-1 MRNA FOR PUTATIVE NUCLEIC ACID BINDING | 252514 | 0.234 |
| | | PROTEIN | | |
| 7048 | 59105 | | 213483 | 0.233 |
| 4472 | 18309 | ESTS | 321723 | 0.23 |
| 7009 | 3701 | PHOSPHOENOLPYRUVATE CARBOXYKINASE, CYTOSOLIC | 187389 | 0.227 |
| 6720 | 139380 | 1 RECORD MOVED FROM CLUSTER 57268 | 85702 | 0.227 |
| 1640 | 4714 | THYROID HORMONE-INDUCIBLE HEPATIC PROTEIN | 82067 | 0.219 |
| 947 | 24443 | ESTS, MODERATELY SIMILAR TO CROOKED NECK PROTEIN | 40022 | 0.214 |
| | | [DROSOPHILA MELANOGASTER] | | |
| 7398 | 9723 | HCG V | 488588 | 0.212 |
| 2460 | 5295 | ZINC FINGER X-CHROMOSOMAL PROTEIN | 140771 | 0.211 |
| 5814 | 32379 | ESTS, WEAKLY SIMILAR TO BRACHYURY PROTEIN | 546980 | 0.196 |
| | | HOMOLOG 2 [BRANCHIOSTOMA FLORIDAE] | | |
| 5527 | 30260 | ESTS, HIGHLY SIMILAR TO 50S RIBOSOMAL PROTEIN L2 | 510950 | 0.196 |
| | | [BACILLUS STEAROTHERMOPHILUS] | | |
| 6939 | 16019 | CYCLIN A1 | 46616 | 0.195 |
| 2865 | 10797 | NF-AT3 | 163576 | 0.184 |
| 4101 | 158888 | 1 RECORD MOVED FROM CLUSTER 3097 | 287851 | 0.154 |
| 1113 | 16331 | GU BINDING PROTEIN | 45187 | 0.133 |
| 2878 | 74204 | SIGMA 3B PROTEIN | 166044 | 0.125 |
| 1455 | 1893 | GLYCINE AMIDINOTRANSFERASE | 74021 | 0.085 |
| 6907 | 5098 | VACUOLAR ATP SYNTHASE SUBUNIT B, KIDNEY ISOFORM | 156211 | 0.071 |
| 6814 | 133728 | 12 RECORDS MOVED FROM CLUSTER 38279 | 38987 | 0.053 |
| 1129 | 251 | ACTIVATOR 1 37 KD SUBUNIT | 46022 | 0.041 |

TABLE 1B

| LifeSpan | LifeSpan | | Image | Bald |
|---|---|---|---|---|
| HDA ID | ClusterID | LifeSpan Cluster Name | CloneID | transit ratio |
| 6287 | 492 | APOLIPOPROTEIN E RECEPTOR 2 | 649804 | 13.903 |
| 2999 | 56337 | E. COLI GENOMIC DNA, KOHARA CLONE #272 (32.4-32.7 MIN.) | 179902 | 7.57 |
| 764 | 23011 | ESTS, HIGHLY SIMILAR TO NADH-CYTOCHROME B5 | 31831 | 7.258 |
| | | REDUCTASE [BOS TAURUS] | | |
| 1193 | 8977 | KIAA0241 | 49139 | 5.299 |
| 721 | 3152 | MICROTUBULE-ASSOCIATED PROTEIN 1B | 29706 | 4.714 |
| 2396 | 1880 | GLUTATHIONE S-TRANSFERASE MU 3 | 137940 | 4.665 |
| 598 | 16147 | GUANINE NUCLEOTIDE REGULATORY FACTOR (LFP40) | 25328 | 4.51 |
| 2959 | 478 | APICAL-LIKE PROTEIN | 175763 | 4.298 |
| 3999 | 28724 | ESTS | 277740 | 4.281 |
| 5498 | 1581 | EUKARYOTIC TRANSLATION INITIATION FACTOR 3 BETA | 510245 | 4 |
| | | SUBUNIT | | |
| 5150 | 154265 | ERBA-RELATED PROTEIN EAR-3 | 471889 | 3.886 |
| 5202 | 2815 | KI NUCLEAR AUTOANTIGEN | 486060 | 3.854 |
| 4373 | 30170 | ESTS, HIGHLY SIMILAR TO HYPOTHETICAL 33.8 KD | 305967 | 3.561 |
| | | PROTEIN R10E11.4 IN CHROMOSOME III [CAENORHABDITIS | | |
| | | ELEGANS] | | |
| 5148 | 7509 | TSPAN-2 | 471879 | 3.291 |
| 1869 | 15344 | TRANSCRIPTIONAL REPRESSOR (NAB1) NAB1 | 113941 | 3.27 |
| 6101 | 24540 | ESTS, HIGHLY SIMILAR TO PROBABLE 26S PROTEASE | 594796 | 3.165 |
| | | SUBUNIT YTA6 [SACCHAROMYCES CEREVISIAE] | | |
| 3477 | 4064 | PROTO-ONCOGENE TYROSINE-PROTEIN KINASE FYN | 232949 | 3.112 |
| 2836 | 178848 | 1 RECORD MOVED FROM CLUSTER 825 | 160729 | 3.107 |
| 6802 | 150811 | 2 RECORDS MOVED FROM CLUSTER 148964 | 35084 | 3.038 |
| 3851 | 4024 | PROTEIN-LYSINE 6-OXIDASE | 266285 | 2.916 |
| 7211 | 822 | CAMP-DEPENDENT PROTEIN KINASE TYPE II-BETA | 310390 | 2.874 |
| | | REGULATORY CHAIN | | |
| 6258 | 17996 | ESTS, WEAKLY SIMILAR TO WEAK SIMILARITY TO RAT | 645512 | 2.843 |
| | | TEGT PROTEIN [C. ELEGANS] | | |
| 2673 | 56267 | ANTIGEN NY-CO-31 (NY-CO-31) | 152748 | 2.806 |
| 4251 | 483 | APOLIPOPROTEIN AI REGULATORY PROTEIN-1 | 298213 | 2.805 |
| 1178 | 23290 | PHOSPHOGLUCOMUTASE-RELATED PROTEIN (PGMRP) | 48182 | 2.671 |
| 6563 | 142186 | 85 RECORDS MOVED FROM CLUSTER 139817 | 757060 | 2.664 |
| 3283 | 5306 | ZONA PELLUCIDA SPERM-BINDING PROTEIN 3A | 206719 | 2.65 |
| 1268 | 4625 | T-CELL RECEPTOR GAMMA CHAIN | 66322 | 2.627 |
| 6910 | 56102 | ALPHA-FETOPROTEIN ENHANCER BINDING PROTEIN | 158983 | 2.581 |
| 6353 | 4494 | SP140 PROTEIN | 685121 | 2.539 |
| 5654 | 139136 | 1 RECORD MOVED FROM CLUSTER 56932 | 530375 | 2.538 |
| 5379 | 1870 | GLUTAREDOXIN | 501952 | 2.506 |
| 7591 | 718 | BRCA2 | 711698 | 2.484 |
| 946 | 4335 | SERINE HYDROXYMETHYLTRANSFERASE, CYTOSOLIC | 39798 | 2.479 |
| 3007 | 14595 | ARGBPIB PROTEIN | 180813 | 2.472 |
| 1764 | 2664 | INTERCELLULAR ADHESION MOLECULE-3 | 109950 | 2.337 |
| 6508 | 3780 | PLECTIN | 741727 | 2.333 |
| 5151 | 41510 | DNA EXCISION REPAIR PROTEIN ERCC-1 | 471895 | 2.318 |
| 4559 | 30499 | EST, WEAKLY SIMILAR TO DYNACTIN, 150 KD ISOFORM | 324510 | 2.312 |
| | | [RATTUS NORVEGICUS] | | |
| 4144 | 119463 | 1 RECORD MOVED FROM CLUSTER 18902 | 290420 | 2.311 |
| 2158 | 2778 | JUNCTION PLAKOGLOBIN | 126320 | 2.302 |
| 4037 | 2089 | HETEROGENEOUS NUCLEAR RIBONUCLEOPROTEIN L | 280298 | 2.3 |
| 5225 | 4329 | SEMAPHORIN E | 486591 | 2.295 |
| 7222 | 38088 | ESTS | 323396 | 2.272 |
| 4766 | 18 | PHOSPHOLIPASE C, GAMMA 1 | 362553 | 2.269 |
| 4703 | 139828 | 4 RECORDS MOVED FROM CLUSTER 77323 | 360595 | 2.267 |
| 5927 | 16097 | INTEGRIN BINDING PROTEIN DEL-1, Z20 SPLICE VARIANT, | 563546 | 2.248 |
| | | (DEL1) | | |
| 1160 | 10935 | INDUCIBLE PROTEIN | 47475 | 2.245 |
| 703 | 96828 | 5 RECORDS MOVED FROM CLUSTER 3380 | 29363 | 2.243 |
| 4244 | 5004 | UBIQUITIN-CONJUGATING ENZYME E2-17 KD UBCC | 297977 | 2.234 |
| 6771 | 58852 | | 129773 | 2.22 |
| 395 | 35402 | DNA FROM CHROMOSOME 19-COSMID R30879 CONTAINING | 511615 | 2.219 |
| | | USF2, GENOMIC | | |
| 5286 | 2826 | KIAA0057 | 488494 | 2.212 |
| 6080 | 155858 | 1 RECORD MOVED FROM CLUSTER 750 | 593166 | 2.207 |
| 5136 | 19896 | ESTS, HIGHLY SIMILAR TO FIBULIN-1, ISOFORM C | 471596 | 2.187 |
| | | PRECURSOR [MUS MUSCULUS] | | |
| 2857 | 4710 | ERBA RELATED PROTEIN EAR-1 | 162751 | 2.176 |
| 4292 | 2994 | M-PROTEIN; skeletal muscle 165 kD protein | 300219 | 2.173 |
| 969 | 41617 | ESTS | 40689 | 2.169 |
| 3394 | 4218 | PHOSPHODIESTERASE PDE6G | 219980 | 2.168 |
| 1046 | 3540 | ONCONEURAL VENTRAL ANTIGEN-1 | 37855 | 2.16 |
| 1309 | 3287 | MYOSIN LIGHT CHAIN ALKALI, SMOOTH-MUSCLE ISOFORM | 68163 | 2.139 |
| 2900 | 3382 | NEURON SPECIFIC CALCIUM-BINDING PROTEIN | 171936 | 2.125 |
| | | HIPPOCALCIN | | |
| 5410 | 3926 | Prostaglandin E2 receptor ep3 | 503146 | 2.123 |
| 3780 | 12298 | AGRIN | 261763 | 2.123 |
| 6878 | 20680 | ESTS | 142969 | 2.119 |
| 1758 | 135296 | 3 RECORDS MOVED FROM CLUSTER 40891 | 109841 | 2.117 |
| 3401 | 10675 | XERODERMA PIGMENTOSUM GROUP E UV-DAMAGED DNA | 220559 | 2.111 |
| | | BINDING FACTOR | | |
| 3125 | 99235 | 1 RECORD MOVED FROM CLUSTER 4227 | 194837 | 2.065 |
| 5591 | 2891 | LAMININ BETA-3 CHAIN | 526215 | 2.061 |
| 6638 | 1384 | DNA-DIRECTED RNA POLYMERASES I, II, AND III 17.1 KD | 781743 | 2.045 |
| | | POLYPEPTIDE | | |
| 7615 | 1357 | DNA TOPOISOMERASE III | 122240 | 2.039 |
| 4236 | 7575 | STEROID RECEPTOR COACTIVATOR-1 | 297675 | 2.035 |
| 2527 | 5269 | ZINC FINGER PROTEIN 83 | 144951 | 2.021 |
| 1971 | 3699 | PHOSPHATIDYLSERINE SYNTHASE I | 118018 | 2.017 |
| 1232 | 18656 | ESTS, HIGHLY SIMILAR TO CMP-N-ACETYLNEURAMINATE- | 51210 | 2.002 |
| | | BETA-1,4-GALACTOSIDE ALPHA-2,3-SIALYLTRANSFERASE | | |
| | | [RATTUS NORVEGICUS] | | |
| 7240 | 4844 | TRANSFORMING GROWTH FACTOR BETA 2 | 326155 | 2.001 |
| 5568 | 26021 | ESTS, WEAKLY SIMILAR TO ZINC FINGER PROTEIN MFG1 | 525319 | 0.497 |
| | | [MUS MUSCULUS] | | |
| 697 | 16603 | CX3C CHEMOKINE | 29324 | 0.497 |
| 6341 | 833 | Cannabinoid receptor 2 | 683442 | 0.496 |
| 3142 | 138665 | 1 RECORD MOVED FROM CLUSTER 56380 | 195643 | 0.496 |
| 5328 | 1046 | COLLAGEN ALPHA 1 (XVIII) CHAIN | 489524 | 0.496 |
| 4105 | 1940 | GRAVE'S DISEASE CARRIER PROTEIN | 288858 | 0.494 |
| 649 | 717 | BRANCHED-CHAIN AMINO ACID AMINOTRANSFERASE, | 27710 | 0.493 |
| | | MITOCHONDRIAL | | |
| 1782 | 151491 | 4 RECORDS MOVED FROM CLUSTER 151026 | 110559 | 0.492 |
| 5278 | 39515 | ESTS, HIGHLY SIMILAR TO COFILIN, MUSCLE ISOFORM | 488362 | 0.492 |
| | | [MUS MUSCULUS] | | |
| 337 | 34869 | ANNEXIN VI | 365553 | 0.492 |
| 1116 | 65 | 3-KETOACYL-COA THIOLASE, MITOCHONDRIAL | 45376 | 0.492 |
| 4384 | 125701 | 5 RECORDS MOVED FROM CLUSTER 23309 | 306222 | 0.491 |
| 470 | 3972 | PROTEIN FARNESYLTRANSFERASE ALPHA SUBUNIT | 21733 | 0.489 |
| 5771 | 4400 | SIGNAL RECOGNITION PARTICLE 54 KD PROTEIN | 545484 | 0.489 |
| 7621 | 566 | ATP-DEPENDENT RNA HELICASE A | 30626 | 0.488 |
| 1444 | 14723 | SELENIUM-BINDING PROTEIN (HSBP) | 73737 | 0.488 |
| 4285 | 2681 | INTERFERON GAMMA UP-REGULATED I-5111 PROTEIN | 300029 | 0.486 |
| 286 | 19096 | ESTS, WEAKLY SIMILAR TO DIAPHANOUS PROTEIN | 298048 | 0.483 |
| | | [D. MELANOGASTER] | | |
| 654 | 3368 | NEUROENDOCRINE PROTEIN 7B2 | 27567 | 0.482 |
| 4725 | 4584 | SYNAPTOSOMAL ASSOCIATED PROTEIN 25 | 361174 | 0.482 |
| 4667 | 16299 | RNASEP PROTEIN P38 (RPP38) | 358044 | 0.481 |
| 2343 | 2311 | HOMEOBOX PROTEIN HOX-B5 | 135050 | 0.481 |
| 4188 | 10671 | P162 | 293940 | 0.479 |
| 651 | 118544 | 75 RECORDS MOVED FROM CLUSTER 18302 | 27635 | 0.479 |
| 3927 | 138855 | 1 RECORD MOVED FROM CLUSTER 56595 | 270692 | 0.479 |
| 1319 | 1574 | EUKARYOTIC INITIATION FACTOR 4A-II | 68591 | 0.479 |
| 495 | 39554 | ESTS | 22242 | 0.478 |
| 6337 | 4404 | SIGNAL TRANSDUCER AND ACTIVATOR OF | 682770 | 0.474 |
| | | TRANSCRIPTION 2 | | |
| 4874 | 56805 | | 415532 | 0.474 |
| 5611 | 56906 | | 526956 | 0.473 |
| 5366 | 181735 | ALPHA-1 CHAIN OF COLLAGEN IV | 491692 | 0.473 |
| 6820 | 4364 | SERINE/THREONINE PROTEIN KINASE PRP4 HOMOLOG | 40240 | 0.471 |
| 1858 | 6350 | MYO-INOSITOL MONOPHOSPHATASE 2 | 113566 | 0.469 |
| 3440 | 17555 | GAMMA-ADAPTIN | 229609 | 0.469 |
| 5001 | 514 | ARFAPTIN 2 | 429547 | 0.469 |
| 1119 | 56837 | BCL7A PROTEIN | 45604 | 0.469 |
| 960 | 3473 | NUCLEAR HORMONE RECEPTOR NOR-1 | 40831 | 0.467 |
| 648 | 119565 | 26 RECORDS MOVED FROM CLUSTER 18987 | 27494 | 0.467 |
| 3834 | 8411 | PROLIFERATING CELL NUCLEAR ANTIGEN | 265407 | 0.464 |
| 3500 | 8330 | PROFILIN I | 235123 | 0.464 |
| 4625 | 2917 | LEUKOCYTE ELASTASE INHIBITOR | 338736 | 0.464 |
| 264 | 918 | CD81 ANTIGEN | 118918 | 0.463 |
| 1280 | 57134 | | 66546 | 0.461 |
| 801 | 138969 | 1 RECORD MOVED FROM CLUSTER 56728 | 33727 | 0.46 |
| 5751 | 57005 | DIAZEPAM-BINDING INHIBITOR | 545081 | 0.459 |
| 3242 | 3281 | MYOSIN LIGHT CHAIN 1, EMBRYONIC MUSCLE/ATRIAL | 203917 | 0.459 |
| | | ISOFORM | | |
| 6572 | 26619 | ESTS | 758420 | 0.459 |
| 4690 | 134834 | 3 RECORDS MOVED FROM CLUSTER 40139 | 360247 | 0.459 |
| 693 | 4511 | SPHAR CYCLIN-RELATED PROTEIN | 29089 | 0.458 |
| 156 | 125 | 5-AMINOLEVULINIC ACID SYNTHASE MITOCHONDRIAL | 22940 | 0.458 |
| | | PRECURSOR, NONSPECIFIC | | |
| 3920 | 183102 | 1 RECORD MOVED FROM CLUSTER 13203 | 270493 | 0.456 |
| 5742 | 3221 | MULTIFUNCTIONAL AMINOACYL-tRNA SYNTHETASE | 544986 | 0.456 |
| 1477 | 1583 | EUKARYOTIC TRANSLATION INITIATION FACTOR 4B | 75035 | 0.453 |
| 3970 | 129480 | 28 RECORDS MOVED FROM CLUSTER 26822 | 274405 | 0.452 |
| 3991 | 4285 | RRP5 PROTEIN HOMOLOG | 277047 | 0.452 |
| 6478 | 2818 | KIAA0027 | 730828 | 0.452 |
| 6560 | 2873 | LACTASE-PHLORIZIN HYDROLASE | 756878 | 0.452 |
| 1704 | 486 | APOLIPOPROTEIN C-I | 85916 | 0.452 |
| 6367 | 222 | CALCIUM-ACTIVATED POTASSIUM CHANNEL SKCA3 | 700710 | 0.451 |
| 5299 | 4043 | PROTEIN TYROSINE PHOSPHATASE MEG2 | 488664 | 0.451 |
| 5186 | 4581 | SYNAPTOBREVIN-LIKE PROTEIN 1 | 485712 | 0.45 |
| 3456 | 89 | RIBOSOMAL PROTEIN S19 | 230363 | 0.449 |
| 6091 | 6168 | SMAD5 | 594181 | 0.448 |
| 5288 | 18273 | ESTS, HIGHLY SIMILAR TO PROBABLE | 488507 | 0.448 |
| | | PHOSPHATIDYLINOSITOL-4-PHOSPHATE 5-KINASE FAB1 | | |
| | | [SACCHAROMYCES CEREVISIAE] | | |
| 7115 | 3693 | PHOSPHATIDYLINOSITOL-4-PHOSPHATE 5-KINASE TYPE II | 267386 | 0.447 |
| 6137 | 1008 | CLEAVAGE STIMULATION FACTOR 77KDA SUBUNIT | 610950 | 0.447 |
| 3065 | 59019 | | 190103 | 0.445 |
| 6430 | 1467 | ELL PROTEIN | 726727 | 0.445 |
| 5578 | 183 | RIBOSOMAL PROTEIN L3-LIKE | 525699 | 0.445 |
| 997 | 22310 | ESTS | 41670 | 0.445 |
| 7264 | 1257 | DEATH-ASSOCIATED PROTEIN KINASE 1 | 341971 | 0.445 |
| 2972 | 1993 | GUANINE NUCLEOTIDE-BINDING PROTEIN G (OLF), ALPHA | 177662 | 0.444 |
| | | SUBUNIT | | |
| 7093 | 3831 | PREGNANCY SPECIFIC BETA-1-GLYCOPROTEIN 4 (PSG4) | 259818 | 0.44 |
| 6712 | 57257 | SHIGELLA FLEXNERI AMINO ACID ANTIPORTER (GADC) | 83500 | 0.437 |
| | | GENE, COMPLETE CDS, AND GLUTAMATE | | |
| | | DECARBOXYLASE (GADB) | | |
| 4742 | 114198 | 76 RECORDS MOVED FROM CLUSTER 14857 | 362023 | 0.437 |
| 7009 | 3701 | PHOSPHOENOLPYRUVATE CARBOXYKINASE, CYTOSOLIC | 187389 | 0.437 |
| 524 | 120979 | 1 RECORD MOVED FROM CLUSTER 20069 | 22750 | 0.437 |
| 5044 | 3922 | PROSTACYCLIN SYNTHASE | 469275 | 0.436 |
| 6111 | 1443 | EARLY ACTIVATION ANTIGEN CD69 | 595331 | 0.436 |
| 6249 | 1726 | G protein-coupled receptor rdc1 | 645026 | 0.434 |
| 2010 | 9023 | CEREBROSIDE SULFOTRANSFERASE | 120278 | 0.434 |
| 717 | 422 | AMILORIDE-SENSITIVE SODIUM CHANNEL BNAC1 | 29692 | 0.434 |
| 5655 | 3573 | OSTEONIDOGEN | 530443 | 0.432 |
| 4769 | 5145 | VOLTAGE-GATED POTASSIUM CHANNEL PROTEIN KV1.1 | 362585 | 0.432 |
| 2504 | 4944 | Angiotensin II type 1a receptor | 143073 | 0.43 |
| 6785 | 22928 | ESTS | 29630 | 0.429 |
| 7107 | 138845 | 1 RECORD MOVED FROM CLUSTER 56576 | 264512 | 0.428 |
| 6104 | 4707 | THYMOSIN BETA-4 | 594922 | 0.428 |
| 2736 | 1374 | DNA-DIRECTED RNA POLYMERASE II 13.3 KD POLYPEPTIDE | 154544 | 0.427 |
| 4758 | 33082 | SERINE/THREONINE PROTEIN KINASE | 362359 | 0.427 |
| 569 | 1564 | ETS TRANSLOCATION VARIANT 1 | 24541 | 0.426 |
| 3405 | 8707 | ROD PHOTORECEPTOR PROTEIN | 221076 | 0.425 |
| 6059 | 4756 | TRANSCOBALAMIN I | 592243 | 0.424 |
| 615 | 16397 | CLONE 23575 | 26451 | 0.422 |
| 1615 | 139370 | 1 RECORD MOVED FROM CLUSTER 57252 | 80790 | 0.422 |
| 4087 | 38965 | SM PROTEIN F | 284538 | 0.419 |
| 3117 | 56372 | | 194484 | 0.419 |
| 4172 | 138901 | 1 RECORD MOVED FROM CLUSTER 56637 | 292434 | 0.417 |
| 3230 | 2067 | HEPATOCYTE GROWTH FACTOR RECEPTOR | 202615 | 0.415 |
| 2534 | 1793 | GAMMA-INTERFERON-INDUCIBLE PROTEIN IFI-16 | 145130 | 0.414 |
| 4948 | 4885 | TROPOMODULIN | 428146 | 0.409 |
| 4657 | 56133 | PROCOLLAGEN ALPHA 2 (IV) CHAIN | 347332 | 0.409 |
| 1074 | 128390 | 1 RECORD MOVED FROM CLUSTER 25647 | 43766 | 0.409 |
| 2045 | 122093 | 1 RECORD MOVED FROM CLUSTER 20688 | 121628 | 0.408 |
| 7086 | 457 | ANNEXIN III | 258375 | 0.408 |
| 6296 | 157070 | 1 RECORD MOVED FROM CLUSTER 1943 | 650445 | 0.405 |
| 927 | 2608 | IMPORTIN BETA-2 SUBUNIT | 39149 | 0.401 |
| 5790 | 1592 | EXCITATORY AMINO ACID TRANSPORTER 3 | 546398 | 0.4 |
| 1390 | 8548 | CH-TOG PROTEIN | 71657 | 0.399 |
| 2319 | 34853 | ESTS, WEAKLY SIMILAR TO RNA-BINDING PROTEIN PIPPIN | 133479 | 0.399 |
| | | [R. NORVEGICUS] | | |
| 1469 | 2360 | HSC70-INTERACTING PROTEIN | 74667 | 0.398 |
| 7177 | 2684 | INTERFERON REGULATORY FACTOR 2 | 297098 | 0.398 |
| 1525 | 679 | BIOTINIDASE | 77938 | 0.398 |
| 6781 | 3218 | MULTIDRUG RESISTANCE PROTEIN 3 | 28573 | 0.395 |
| 4434 | 2740 | INTERLEUKIN-6 | 310406 | 0.394 |
| 5308 | 7348 | NADH-UBIQUINONE OXIDOREDUCTASE SUBUNIT CI-SGDH | 488830 | 0.394 |
| 1558 | 40103 | ESTS, MODERATELY SIMILAR TO ANTIFREEZE | 79146 | 0.393 |
| | | GLYCOPEPTIDE POLYPROTEIN AFGP7/AFGP8 PRECURSOR | | |
| | | [NOTOTHENIA CORIICEPS NEGLECTA] | | |
| 7622 | 1133 | CYCLIN G | 32322 | 0.392 |
| 808 | 633 | BETA GALACTOSIDASE-RELATED PROTEIN | 33943 | 0.392 |
| 630 | 4650 | T54 PROTEIN | 26910 | 0.39 |
| 6958 | 39960 | ESTS | 51015 | 0.389 |
| 6783 | 160624 | UNC-51-LIKE KINASE ULK1 | 28732 | 0.389 |
| 5364 | 28805 | ESTS | 491495 | 0.388 |
| 3187 | 2068 | HEPATOCYTE GROWTH FACTOR-LIKE PROTEIN | 198656 | 0.388 |
| 3773 | 9677 | PREGNANCY-SPECIFIC BETA-1-GLYCOPROTEIN PSG95 | 260126 | 0.386 |
| 1161 | 143112 | 66 RECORDS MOVED FROM CLUSTER 142487 | 47795 | 0.386 |
| 497 | 3276 | MYOSIN HEAVY CHAIN, NONMUSCLE TYPE A | 22140 | 0.385 |
| 3918 | 11020 | ENDOGENOUS RETROVIRUS TYPE C | 270385 | 0.38 |
| 5146 | 156057 | 1 RECORD MOVED FROM CLUSTER 855 | 471861 | 0.38 |
| 3930 | 8962 | KIAA0266 | 270753 | 0.378 |
| 4312 | 100432 | 1 RECORD MOVED FROM CLUSTER 4777 | 301162 | 0.377 |
| 7356 | 123717 | 4 RECORDS MOVED FROM CLUSTER 21938 | 416093 | 0.377 |
| 1378 | 2642 | INSULIN-LIKE GROWTH FACTOR II | 71160 | 0.373 |
| 4514 | 5190 | XE169 PROTEIN | 322859 | 0.37 |
| 3908 | 138851 | 1 RECORD MOVED FROM CLUSTER 56588 | 269809 | 0.369 |
| 2102 | 2115 | HIGH MOBILITY GROUP PROTEIN HMG2 | 124257 | 0.368 |
| 641 | 11193 | MYELIN PROTEOLIPID PROTEIN | 27308 | 0.368 |
| 2865 | 10797 | NF-AT3 | 163576 | 0.366 |
| 3892 | 817 | PHOSPHODIESTERASE PDE4D | 268455 | 0.366 |
| 4209 | 1174 | CYTOCHROME B561 | 295787 | 0.361 |
| 604 | 22592 | ESTS, HIGHLY SIMILAR TO FIBROPELLIN C PRECURSOR | 25810 | 0.359 |
| | | [STRONGYLOCENTROTUS PURPURATUS] | | |
| 5739 | 56999 | | 544952 | 0.358 |
| 6729 | 138356 | 1 RECORD MOVED FROM CLUSTER 56108 | 110005 | 0.355 |
| 4765 | 31344 | | 362530 | 0.354 |
| 6720 | 139380 | 1 RECORD MOVED FROM CLUSTER 57268 | 85702 | 0.353 |
| 698 | 1288 | DEVELOPMENTALLY REGULATED GTP-BINDING PROTEIN | 29328 | 0.347 |
| | | DRG | | |
| 845 | 13531 | ALPHA1-SYNTROPHIN (SNT A1) | 35230 | 0.343 |
| 536 | 8668 | TRANSLATION INITIATION FACTOR 3 LARGE SUBUNIT | 23348 | 0.337 |
| 5740 | 7020 | MACROPHAGE RECEPTOR MARCO | 544976 | 0.336 |
| 2048 | 1638 | FIBRILLIN 2 | 121722 | 0.334 |
| 6939 | 16019 | CYCLIN A1 | 46616 | 0.333 |
| 6260 | 5071 | URIDINE DIPHOSPHOGLUCOSE PYROPHOSPHORYLASE | 646513 | 0.333 |
| 7456 | 56908 | BETA-DEFENSIN 1 | 527061 | 0.332 |
| 2130 | 594 | B-LYMPHOCYTE ACTIVATION MARKER BLAST-1 | 125134 | 0.331 |
| 7461 | 59620 | | 531428 | 0.331 |
| 4984 | 1977 | GUANINE NUCLEOTIDE EXCHANGE FACTOR PROTEIN TRIO | 429234 | 0.33 |
| 2713 | 33116 | ESTS | 154032 | 0.329 |
| 6150 | 57105 | TITIN Z-DISC | 611590 | 0.328 |
| 7470 | 57018 | | 545704 | 0.326 |
| 1370 | 308 | ADRENAL SPECIFIC 30 KD PROTEIN | 70777 | 0.321 |
| 2897 | 15388 | M. FASCICULARIS MRNA FOR NAD+−ISOCITRATE | 171786 | 0.32 |
| | | DEHYDROGENASE | | |
| 7017 | 135729 | 1 RECORD MOVED FROM CLUSTER 44399 | 193496 | 0.318 |
| 6420 | 610 | BASIC TRANSCRIPION FACTOR 2, 44 KD SUBUNIT | 726071 | 0.316 |
| 2128 | 79132 | 1 RECORD MOVED FROM CLUSTER 637 | 125092 | 0.311 |
| 7091 | 7549 | CLONE 24684 | 259162 | 0.31 |
| 5036 | 1837 | GLUCOCORTICOID RECEPTOR REPRESSION FACTOR 1 | 430335 | 0.303 |
| 6093 | 477 | AP-2 GAMMA TRANSCRIPTION FACTOR | 594372 | 0.302 |
| 2984 | 26367 | ESTS, HIGHLY SIMILAR TO HYPOTHETICAL 56.5 KD | 178459 | 0.3 |
| | | PROTEIN IN DYS1-ERG7 INTERGENIC REGION | | |
| | | [SACCHAROMYCES CEREVISIAE] | | |
| 535 | 18541 | ESTS | 23141 | 0.299 |
| 2570 | 8798 | E14 PROTEIN; NPAT | 146987 | 0.299 |
| 1367 | 2381 | HYPOTHETICAL 33.4 KD PROTEIN | 70627 | 0.292 |
| 7479 | 3685 | PHOSPHATIDYLINOSITOL 3-KINASE | 549264 | 0.29 |
| 5527 | 30260 | ESTS, HIGHLY SIMILAR TO 50S RIBOSOMAL PROTEIN L2 | 510950 | 0.29 |
| | | [BACILLUS STEAROTHERMOPHILUS] | | |
| 7623 | 1343 | DNA POLYMERASE GAMMA | 32577 | 0.288 |
| 5340 | 56856 | ESTS, WEAKLY SIMILAR TO HYPOTHETICAL 32.0 KD | 489814 | 0.278 |
| | | PROTEIN IN SAP190-SPO14 INTERGENIC REGION | | |
| | | [SACCHAROMYCES CEREVISIAE] | | |
| 238 | 601 | B4-2 PROTEIN | 61261 | 0.276 |
| 2967 | 4809 | TRANSCRIPTION INITIATION FACTOR IIF, ALPHA SUBUNIT | 177140 | 0.275 |
| 406 | 4682 | THIOL-SPECIFIC ANTIOXIDANT | 531464 | 0.275 |
| 7154 | 28928 | ESTS | 281041 | 0.27 |
| 2612 | 1485 | ENDOTHELIAL TRANSCRIPTION FACTOR GATA-2 | 149809 | 0.268 |
| 6512 | 505 | AQUAPORIN 5 | 741914 | 0.268 |
| 3655 | 2819 | KIAA0110 | 252258 | 0.266 |
| 5514 | 732 | BYSTIN | 510607 | 0.257 |
| 3943 | 11984 | CDC2-RELATED PROTEIN KINASE CHED | 271662 | 0.252 |
| 973 | 96833 | 3 RECORDS MOVED FROM CLUSTER 3386 | 41074 | 0.245 |
| 7294 | 56742 | | 346081 | 0.236 |
| 4837 | 1862 | GLUTAMATE RECEPTOR 7 | 381812 | 0.235 |
| 6716 | 801 | CALPAIN 1, LARGE | 84298 | 0.232 |
| 216 | 4961 | TYROSINE-PROTEIN KINASE LYN | 196007 | 0.228 |
| 1000 | 1042 | COLLAGEN ALPHA 1 (XI) CHAIN | 41676 | 0.226 |
| 7048 | 59105 | | 213483 | 0.225 |
| 3662 | 30158 | RY-1 MRNA FOR PUTATIVE NUCLEIC ACID BINDING | 252514 | 0.223 |
| | | PROTEIN | | |
| 1492 | 59871 | | 75898 | 0.221 |
| 4101 | 158888 | 1 RECORD MOVED FROM CLUSTER 3097 | 287851 | 0.206 |
| 5536 | 5233 | ZINC FINGER PROTEIN 191 | 511410 | 0.204 |
| 733 | 21908 | ESTS, HIGHLY SIMILAR TO UTR4 PROTEIN | 30452 | 0.201 |
| | | [SACCHAROMYCES CEREVISIAE] | | |
| 2460 | 5295 | ZINC FINGER X-CHROMOSOMAL PROTEIN | 140771 | 0.2 |
| 486 | 56048 | ESTS, HIGHLY SIMILAR TO DNA-BINDING PROTEIN SATB1 | 21969 | 0.199 |
| | | [HOMO SAPIENS] | | |
| 5084 | 18785 | RAD1 | 470124 | 0.198 |
| 7480 | 19495 | RATTUS NORVEGICUS MAXP1 | 550298 | 0.195 |
| 3400 | 3618 | PAIRED BOX PROTEIN PAX-6 | 220373 | 0.189 |
| 702 | 5019 | UDP-GALACTOSE TRANSLOCATOR | 29362 | 0.17 |
| 2014 | 1214 | CYTOCHROME P450 IVA11 | 120466 | 0.17 |
| 4628 | 3042 | MATRILYSIN | 338835 | 0.168 |
| 1183 | 2779 | POTASSIUM CHANNEL BETA 1A SUBUNIT | 48631 | 0.161 |
| 1126 | 4593 | SYNTAXIN 3 | 45789 | 0.156 |
| 4333 | 15747 | SER-THR PROTEIN KINASE PK428 | 302177 | 0.154 |
| 1006 | 3592 | P2X4 ATP-GATED CATION CHANNEL PROTEIN | 42118 | 0.141 |
| 1640 | 4714 | THYROID HORMONE-INDUCIBLE HEPATIC PROTEIN | 82067 | 0.137 |
| 1611 | 33367 | E-MAP-115 | 80734 | 0.136 |
| 1455 | 1893 | GLYCINE AMIDINOTRANSFERASE | 74021 | 0.135 |
| 4907 | 2083 | INHIBIN BETA C CHAIN | 417539 | 0.129 |
| 7191 | 1890 | GLYCEROL KINASE | 305572 | 0.095 |
| 1032 | 4710 | ERBA RELATED PROTEIN EAR-1 | 42706 | 0.021 |

TABLE 1C

| LifeSpan | LifeSpan | | Image | NonBald |
|---|---|---|---|---|
| HDA ID | Cluster ID | LifeSpan Cluster Name | CloneID | TransitPhase ratio |
| 1129 | 251 | ACTIVATOR 1 37 KD SUBUNIT | 46022 | 24.09 |
| 7397 | 3840 | PROBABLE ACTIN-BINDING PROTEIN ACF7 | 488364 | 16.56 |
| 6907 | 5098 | VACUOLAR ATP SYNTHASE SUBUNIT B, KIDNEY ISOFORM | 156211 | 16.05 |
| 2878 | 74204 | SIGMA 3B PROTEIN | 166044 | 14.746 |
| 6814 | 133728 | 12 RECORDS MOVED FROM CLUSTER 38279 | 38987 | 13.838 |
| 5498 | 1581 | EUKARYOTIC TRANSLATION INITIATION FACTOR 3 BETA | 510245 | 10.611 |
| | | SUBUNIT | | |
| 971 | 6736 | NEUROCAN (CSPG3) | 41261 | 8.31 |
| 2964 | 1542 | ERPROT 213-21 | 176786 | 7.69 |
| 1113 | 16331 | GU BINDING PROTEIN | 45187 | 7.51 |
| 7398 | 9723 | HCG V | 488588 | 7.341 |
| 7334 | 4769 | TRANSCRIPTION FACTOR E2-ALPHA | 366893 | 6.883 |
| 7136 | 5619 | MBNL PROTEIN | 271915 | 6.002 |
| 3283 | 5306 | ZONA PELLUCIDA SPERM-BINDING PROTEIN 3A | 206719 | 5.771 |
| 7591 | 718 | BRCA2 | 711698 | 5.297 |
| 869 | 2902 | LEGUMAIN | 36128 | 4.939 |
| 1682 | 120918 | 34 RECORDS MOVED FROM CLUSTER 20034 | 84191 | 4.89 |
| 3232 | 56404 | T-STAR (T-STAR) | 202836 | 4.846 |
| 2693 | 16820 | NOVEL T-CELL ACTIVATION PROTEIN | 153460 | 4.689 |
| 947 | 24443 | ESTS, MODERATELY SIMILAR TO CROOKED NECK PROTEIN | 40022 | 4.455 |
| | | [DROSOPHILA MELANOGASTER] | | |
| 5148 | 7509 | TSPAN-2 | 471879 | 4.406 |
| 7444 | 5005 | UBIQUITIN-CONJUGATING ENZYME E2-18 KD | 511757 | 4.224 |
| 1045 | 158270 | 1 RECORD MOVED FROM CLUSTER 2345 | 37841 | 4.194 |
| 6258 | 17996 | ESTS, WEAKLY SIMILAR TO WEAK SIMILARITY TO RAT TEGT | 645512 | 4.111 |
| | | PROTEIN [C. ELEGANS] | | |
| 1036 | 790 | CALCYPHOSINE | 42992 | 3.972 |
| 4373 | 30170 | ESTS, HIGHLY SIMILAR TO HYPOTHETICAL 33.8 KD PROTEIN | 305967 | 3.968 |
| | | R10E11.4 IN CHROMOSOME III [CAENORHABDITIS ELEGANS] | | |
| 5814 | 32379 | ESTS, WEAKLY SIMILAR TO BRACHYURY PROTEIN HOMOLOG | 546980 | 3.829 |
| | | 2 [BRANCHIOSTOMA FLORIDAE] | | |
| 5725 | 56985 | | 544693 | 3.715 |
| 4251 | 483 | APOLIPOPROTEIN AI REGULATORY PROTEIN-1 | 298213 | 3.606 |
| 2674 | 18853 | ESTS, WEAKLY SIMILAR TO PRE-MRNA SPLICING FACTOR | 152763 | 3.449 |
| | | SRP75 [HOMO SAPIENS] | | |
| 1160 | 10935 | INDUCIBLE PROTEIN | 47475 | 3.43 |
| 6599 | 4797 | TRANSCRIPTION FACTOR SP2 | 770397 | 3.419 |
| 6754 | 137397 | 1 RECORD MOVED FROM CLUSTER 48960 | 120513 | 3.416 |
| 4131 | 33325 | PHOSPHODIESTERASE PDE8A | 289972 | 3.388 |
| 5808 | 139168 | 1 RECORD MOVED FROM CLUSTER 57032 | 546829 | 3.337 |
| 2979 | 3889 | PROCHOLECYSTOKININ | 178091 | 3.175 |
| 1666 | 1483 | ENDOPLASMIN | 83465 | 3.166 |
| 7406 | 134667 | 37 RECORDS MOVED FROM CLUSTER 39938 | 489983 | 3.142 |
| 6977 | 25933 | ESTS, HIGHLY SIMILAR TO SYNTAXIN A [BOS TAURUS] | 172237 | 3.105 |
| 1869 | 15344 | TRANSCRIPTIONAL REPRESSOR (NAB1) NAB1 | 113941 | 3.07 |
| 2754 | 33493 | 5T4 GENE FOR 5T4 ONCOFETAL ANTIGEN | 155195 | 3.039 |
| 4904 | 135686 | 1 RECORD MOVED FROM CLUSTER 44355 | 417434 | 3.033 |
| 3851 | 4024 | PROTEIN-LYSINE 6-OXIDASE | 266285 | 2.946 |
| 5070 | 417 | ALZHEIMER'S DISEASE AMYLOID A4 PROTEIN | 469840 | 2.924 |
| 1916 | 56077 | Orphan G protein-coupled receptor 56077 | 115277 | 2.858 |
| 6428 | 1944 | GRB14 | 726559 | 2.829 |
| 3008 | 15235 | CDC37 HOMOLOG | 181066 | 2.785 |
| 1519 | 669 | BETAINE-HOMOCYSTEINE S-METHYLTRANSFERASE | 77636 | 2.764 |
| 1178 | 23290 | PHOSPHOGLUCOMUTASE-RELATED PROTEIN (PGMRP) | 48182 | 2.751 |
| 1395 | 1673 | FLI-1 ONCOGENE | 71821 | 2.701 |
| 959 | 4833 | TRANSCRIPTIONAL REPRESSOR NF-X1 | 40265 | 2.662 |
| 4472 | 18309 | ESTS | 321723 | 2.656 |
| 1284 | 2835 | KIAA0099 | 66628 | 2.65 |
| 692 | 3375 | NEUROGENIC LOCUS NOTCH PROTEIN HOMOLOG 1 | 28967 | 2.648 |
| 301 | 401 | ALPHA-L-IDURONIDASE | 325954 | 2.595 |
| 946 | 4335 | SERINE HYDROXYMETHYLTRANSFERASE, CYTOSOLIC | 39798 | 2.589 |
| 4807 | 2785 | KERATIN, TYPE II HAIR-SPECIFIC | 365043 | 2.552 |
| 7163 | 129287 | 1 RECORD MOVED FROM CLUSTER 26618 | 288919 | 2.502 |
| 260 | 1585 | EUKARYOTIC TRANSLATION INITIATION FACTOR 5 | 113597 | 2.488 |
| 4799 | 4158 | RAS-RELATED PROTEIN RAB-1A | 363872 | 2.482 |
| 3106 | 18886 | ESTS | 193900 | 2.427 |
| 6992 | 24195 | ESTS | 177520 | 2.4 |
| 2171 | 5630 | KIAA0439 | 126828 | 2.378 |
| 6520 | 4102 | PUTATIVE REGULATORY PROTEIN TSC-22 | 742674 | 2.373 |
| 5421 | 2386 | HYPOTHETICAL PROTEIN | 503809 | 2.372 |
| 1022 | 3859 | G protein-coupled receptor gpr22 | 42685 | 2.365 |
| 6975 | 25981 | ESTS | 172140 | 2.349 |
| 4697 | 38171 | ESTS, WEAKLY SIMILAR TO TWITCHIN [C. ELEGANS] | 360472 | 2.335 |
| 7546 | 3700 | PHOSPHOENOLPYRUVATE CARBOXYKINASE | 625923 | 2.325 |
| 6627 | 3921 | Prostacyclin receptor | 774146 | 2.32 |
| 4603 | 4856 | TRANSITIONAL ENDOPLASMIC RETICULUM ATPASE | 328401 | 2.3 |
| 6531 | 224 | AC1 | 743212 | 2.291 |
| 773 | 138959 | 1 RECORD MOVED FROM CLUSTER 56691 | 32212 | 2.291 |
| 7399 | 31750 | ESTS | 488683 | 2.29 |
| 4317 | 2083 | INHIBIN BETA C CHAIN | 301507 | 2.284 |
| 1040 | 3024 | MALATE OXIDOREDUCTASE | 42910 | 2.275 |
| 6353 | 4494 | SP140 PROTEIN | 685121 | 2.271 |
| 1109 | 139073 | 2 RECORDS MOVED FROM CLUSTER 56836 | 45153 | 2.225 |
| 5927 | 16097 | INTEGRIN BINDING PROTEIN DEL-1, Z20 SPLICE VARIANT, | 563546 | 2.213 |
| | | (DEL1) | | |
| 4045 | 3937 | PROTACHYKININ BETA | 280510 | 2.187 |
| 5173 | 24237 | ESTS | 485164 | 2.17 |
| 6638 | 1384 | DNA-DIRECTED RNA POLYMERASES I, II, AND III 17.1 KD | 781743 | 2.169 |
| | | POLYPEPTIDE | | |
| 393 | 5021 | UDP-GLUCURONOSYLTRANSFERASE 1-2 PRECURSOR, | 511275 | 2.169 |
| | | MICROSOMAL | | |
| 849 | 1901 | GLYCOGEN (STARCH) SYNTHASE, MUSCLE | 35615 | 2.162 |
| 6979 | 8335 | HETEROGENEOUS NUCLEAR RIBONUCLEOPROTEINS C1/C2 | 172356 | 2.159 |
| 1046 | 3540 | ONCONEURAL VENTRAL ANTIGEN-1 | 37855 | 2.156 |
| 2688 | 5530 | AQUAPORIN-7 LIKE | 153310 | 2.151 |
| 4896 | 141607 | 2 RECORDS MOVED FROM CLUSTER 123787 | 417024 | 2.143 |
| 2742 | 15041 | MATRIX METALLOPROTEINASE MMP-18 | 154770 | 2.14 |
| 4408 | 4739 | TISSUE ALPHA-L-FUCOSIDASE | 308437 | 2.139 |
| 6069 | 1340 | DNA POLYMERASE DELTA SMALL SUBUNIT | 592659 | 2.117 |
| 6563 | 142186 | 85 RECORDS MOVED FROM CLUSTER 139817 | 757060 | 2.104 |
| 5941 | 139180 | 1 RECORD MOVED FROM CLUSTER 57065 | 564205 | 2.102 |
| 5486 | 519 | ARGININOSUCCINATE LYASE | 510018 | 2.099 |
| 945 | 40047 | ESTS, HIGHLY SIMILAR TO LAMBDA-CRYSTALLIN | 40115 | 2.086 |
| | | [ORYCTOLAGUS CUNICULUS] | | |
| 879 | 125640 | 4 RECORDS MOVED FROM CLUSTER 23267 | 36716 | 2.078 |
| 4559 | 30499 | EST, WEAKLY SIMILAR TO DYNACTIN, 150 KD ISOFORM | 324510 | 2.071 |
| | | [RATTUS NORVEGICUS] | | |
| 865 | 3718 | PHOSPHORIBOSYLAMINE--GLYCINE LIGASE | 36299 | 2.064 |
| 1125 | 273 | Adenosine A2a receptor | 45788 | 2.054 |
| 3477 | 4064 | PROTO-ONCOGENE TYROSINE-PROTEIN KINASE FYN | 232949 | 2.048 |
| 171 | 124531 | 17 RECORDS MOVED FROM CLUSTER 22554 | 40160 | 2.03 |
| 6611 | 4851 | TRANSFORMING PROTEIN P21/K-RAS 2B | 773243 | 2.027 |
| 979 | 4627 | T-CELL SURFACE GLYCOPROTEIN CD4 | 41134 | 2.017 |
| 1548 | 748 | C-REACTIVE PROTEIN | 78639 | 2.011 |
| 4710 | 39778 | ESTS, HIGHLY SIMILAR TO SODIUM-INDEPENDENT ORGANIC | 360813 | 2.008 |
| | | ANION TRANSPORTER [RATTUS NORVEGICUS] | | |
| 4831 | 59443 | | 381007 | 2.004 |
| 7047 | 27272 | ESTS | 212252 | 2.003 |
| 3547 | 347 | ALDOSE REDUCTASE | 240140 | 2.001 |
| 5753 | 57006 | | 545097 | 0.497 |
| 972 | 139025 | 1 RECORD MOVED FROM CLUSTER 56797 | 41161 | 0.497 |
| 2549 | 25467 | ESTS, HIGHLY SIMILAR TO RAS-RELATED PROTEIN RAB-12 | 145770 | 0.496 |
| | | [RATTUS NORVEGICUS] | | |
| 1975 | 5940 | ACTIN-BINDING DOUBLE-ZINC-FINGER PROTEIN (ABLIM) | 118588 | 0.496 |
| 4625 | 2917 | LEUKOCYTE ELASTASE INHIBITOR | 338736 | 0.496 |
| 3516 | 4296 | S100 CALCIUM-BINDING PROTEIN A2 | 238479 | 0.495 |
| 6139 | 104942 | 19 RECORDS MOVED FROM CLUSTER 6877 | 611003 | 0.494 |
| 535 | 18541 | ESTS | 23141 | 0.493 |
| 495 | 39554 | ESTS | 22242 | 0.492 |
| 7063 | 4795 | TRANSCRIPTION FACTOR SOX-9 | 240393 | 0.491 |
| 1615 | 139370 | 1 RECORD MOVED FROM CLUSTER 57252 | 80790 | 0.491 |
| 2827 | 3199 | MONOCYTE DIFFERENTIATION ANTIGEN CD14 | 159946 | 0.488 |
| 2984 | 26367 | ESTS, HIGHLY SIMILAR TO HYPOTHETICAL 56.5 KD PROTEIN | 178459 | 0.487 |
| | | IN DYS1-ERG7 INTERGENIC REGION [SACCHAROMYCES | | |
| | | CEREVISIAE] | | |
| 4105 | 1940 | GRAVE'S DISEASE CARRIER PROTEIN | 288858 | 0.485 |
| 7088 | 26722 | ESTS | 258776 | 0.485 |
| 6094 | 15873 | POLY (A)-BINDING PROTEIN | 594452 | 0.484 |
| 5990 | 1623 | FATTY ACID-BINDING PROTEIN, ADIPOCYTE | 567306 | 0.483 |
| 5299 | 4043 | PROTEIN TYROSINE PHOSPHATASE MEG2 | 488664 | 0.483 |
| 2851 | 490 | APOLIPOPROTEIN D | 162182 | 0.481 |
| 7523 | 2790 | KERATIN, TYPE I CYTOSKELETAL 15 | 592758 | 0.481 |
| 6826 | 5391 | A-KINASE ANCHOR PROTEIN (AKAP100) | 40844 | 0.481 |
| 6892 | 2760 | IROQUOIS-CLASS HOMEODOMAIN PROTEIN IRX-1 | 152453 | 0.481 |
| 5181 | 1664 | FIBULIN-2 | 485648 | 0.481 |
| 2301 | 89716 | 143 RECORDS MOVED FROM CLUSTER 1658 | 132559 | 0.479 |
| 4694 | 1369 | DNA-BINDING PROTEIN MEL-18 | 360329 | 0.478 |
| 7264 | 1257 | DEATH-ASSOCIATED PROTEIN KINASE 1 | 341971 | 0.477 |
| 826 | 100275 | 6 RECORDS MOVED FROM CLUSTER 4718 | 34867 | 0.475 |
| 2475 | 638 | BETA-1,4-GALACTOSYLTRANSFERASE | 141570 | 0.475 |
| 5924 | 57063 | ESCHERICHIA COLI GENOMIC DNA. (16.1-16.4 MIN) | 563439 | 0.475 |
| 1478 | 16163 | LOT1 | 75187 | 0.474 |
| 1308 | 33048 | 218KD MI-2 PROTEIN | 68089 | 0.472 |
| 3117 | 56372 | | 194484 | 0.472 |
| 4098 | 29190 | PRR1 | 287663 | 0.47 |
| 2882 | 1851 | N-METHYL-D-ASPARTATE RECEPTOR 1 NMDAR1 | 166245 | 0.469 |
| 156 | 125 | 5-AMINOLEVULINIC ACID SYNTHASE MITOCHONDRIAL | 22940 | 0.464 |
| | | PRECURSOR, NONSPECIFIC | | |
| 5335 | 7645 | ZINC FINGER PROTEIN | 489702 | 0.463 |
| 5507 | 2352 | KIAA0043 | 510388 | 0.463 |
| 997 | 22310 | ESTS | 41670 | 0.463 |
| 7092 | 28276 | EST | 259390 | 0.463 |
| 1391 | 3453 | NON-RECEPTOR TYROSINE-PROTEIN KINASE TYK2 | 71662 | 0.462 |
| 7394 | 4236 | RETINOIC ACID-BINDING PROTEIN II, CELLULAR | 487957 | 0.462 |
| 5602 | 19627 | UNKNOWN | 526496 | 0.461 |
| 7107 | 138845 | 1 RECORD MOVED FROM CLUSTER 56576 | 264512 | 0.459 |
| 461 | 51 | 26S PROTEASE REGULATORY SUBUNIT P42 | 53193 | 0.457 |
| 7346 | 59445 | | 381228 | 0.457 |
| 1525 | 679 | BIOTINIDASE | 77938 | 0.457 |
| 5524 | 693 | BONE MARROW STROMAL ANTIGEN 2 | 510866 | 0.455 |
| 6871 | 39577 | ESTS | 140061 | 0.455 |
| 7385 | 31632 | ESTS | 485905 | 0.452 |
| 1538 | 120194 | 3 RECORDS MOVED FROM CLUSTER 19417 | 78262 | 0.452 |
| 5609 | 17676 | STRATUM CORNEUM CHYMOTRYPTIC ENZYME (SCCE) | 526892 | 0.447 |
| 7480 | 19495 | RATTUS NORVEGICUS MAXP1 | 550298 | 0.445 |
| 2996 | 6908 | VOLTAGE-GATED POTASSIUM CHANNEL KCNQ2 | 179534 | 0.445 |
| 517 | 1995 | GUANINE NUCLEOTIDE-BINDING PROTEIN G (S), ALPHA | 23019 | 0.444 |
| | | SUBUNIT | | |
| 2438 | 3401 | NEURONATIN | 139681 | 0.442 |
| 6140 | 1302 | DIHYDROOROTATE DEHYDROGENASE | 611027 | 0.441 |
| 801 | 138969 | 1 RECORD MOVED FROM CLUSTER 56728 | 33727 | 0.441 |
| 6366 | 148652 | 1 RECORD MOVED FROM CLUSTER 34480 | 700666 | 0.44 |
| 3242 | 3281 | MYOSIN LIGHT CHAIN 1, EMBRYONIC MUSCLE/ATRIAL | 203917 | 0.436 |
| | | ISOFORM | | |
| 1973 | 1063 | COMPLEMENT C1S COMPONENT | 118237 | 0.432 |
| 7154 | 28928 | ESTS | 281041 | 0.43 |
| 6455 | 2619 | INITIATION FACTOR 5A | 728017 | 0.427 |
| 569 | 1564 | ETS TRANSLOCATION VARIANT 1 | 24541 | 0.426 |
| 4480 | 12982 | BRAIN-EXPRESSED HHCPA78 HOMOLOG | 322101 | 0.422 |
| 5102 | 2799 | KERATIN, TYPE II CYTOSKELETAL 2 EPIDERMAL | 470517 | 0.422 |
| 7113 | 5255 | ZINC FINGER PROTEIN 40 | 266854 | 0.421 |
| 4308 | 29917 | UNKNOWN PROTEIN EXPRESSED IN MACROPHAGES | 301018 | 0.42 |
| 5655 | 3573 | OSTEONIDOGEN | 530443 | 0.419 |
| 7329 | 6740 | PUTATIVE ONCOGENE PROTEIN | 366519 | 0.417 |
| 3371 | 1247 | D-BETA-HYDROXYBUTYRATE DEHYDROGENASE | 214108 | 0.416 |
| 3400 | 3618 | PAIRED BOX PROTEIN PAX-6 | 220373 | 0.415 |
| 3513 | 135825 | 1 RECORD MOVED FROM CLUSTER 44497 | 238349 | 0.41 |
| 5659 | 24423 | CYP4A LOCUS, ENCODING CYTOCHROME P450 (IVA3) | 530623 | 0.409 |
| 648 | 119565 | 26 RECORDS MOVED FROM CLUSTER 18987 | 27494 | 0.408 |
| 1611 | 33367 | E-MAP-115 | 80734 | 0.408 |
| 1604 | 802 | CALPAIN 2, LARGE | 80614 | 0.404 |
| 825 | 15211 | RAGA PROTEIN | 34660 | 0.4 |
| 1850 | 843 | CARBONYL REDUCTASE | 113023 | 0.398 |
| 4175 | 4140 | RAN GTPASE ACTIVATING PROTEIN 1 | 292619 | 0.398 |
| 6942 | 3977 | PROTEIN KINASE C DELTA-TYPE | 47306 | 0.395 |
| 5075 | 7117 | 39 KDA PROTEIN | 469977 | 0.394 |
| 7177 | 2684 | INTERFERON REGULATORY FACTOR 2 | 297098 | 0.394 |
| 2047 | 19346 | ESTS, HIGHLY SIMILAR TO COMPLEMENT RECEPTOR TYPE 2 | 121678 | 0.388 |
| | | PRECURSOR [MUS MUSCULUS] | | |
| 2262 | 138554 | 1 RECORD MOVED FROM CLUSTER 56207 | 130506 | 0.383 |
| 1119 | 56837 | BCL7A PROTEIN | 45604 | 0.382 |
| 570 | 64005 | DYSTROBREVIN B DTN-B1 | 24544 | 0.382 |
| 246 | 1194 | CYTOCHROME P450 IVF3 | 78317 | 0.38 |
| 450 | 159972 | | 53024 | 0.378 |
| 7211 | 822 | CAMP-DEPENDENT PROTEIN KINASE TYPE II-BETA | 310390 | 0.377 |
| | | REGULATORY CHAIN | | |
| 3743 | 48224 | MUF1 PROTEIN | 258835 | 0.373 |
| 2673 | 56267 | ANTIGEN NY-CO-31 (NY-CO-31) | 152748 | 0.372 |
| 3834 | 8411 | PROLIFERATING CELL NUCLEAR ANTIGEN | 265407 | 0.371 |
| 5578 | 183 | RIBOSOMAL PROTEIN L3-LIKE | 525699 | 0.371 |
| 1378 | 2642 | INSULIN-LIKE GROWTH FACTOR II | 71160 | 0.369 |
| 3421 | 5767 | ATP-BINDING CASSETTE TRANSPORTER (ABCR) | 222197 | 0.368 |
| 3991 | 4285 | RRP5 PROTEIN HOMOLOG | 277047 | 0.366 |
| 741 | 5661 | SPERM PROTEIN | 30838 | 0.366 |
| 808 | 633 | BETA GALACTOSIDASE-RELATED PROTEIN | 33943 | 0.365 |
| 5260 | 2794 | KERATIN, TYPE I CYTOSKELETAL 18 | 487868 | 0.363 |
| 3440 | 17555 | GAMMA-ADAPTIN | 229609 | 0.359 |
| 7122 | 3276 | MYOSIN HEAVY CHAIN, NONMUSCLE TYPE A | 268282 | 0.358 |
| 711 | 13012 | NF-E2-LIKE BASIC LEUCINE ZIPPER TRANSCRIPTIONAL | 29541 | 0.353 |
| | | ACTIVATOR (NRF2) | | |
| 5611 | 56906 | | 526956 | 0.349 |
| 7300 | 8941 | SERINE PROTEASE | 347396 | 0.345 |
| 7086 | 457 | ANNEXIN III | 258375 | 0.344 |
| 4514 | 5190 | XE169 PROTEIN | 322859 | 0.341 |
| 7166 | 138888 | 1 RECORD MOVED FROM CLUSTER 56625 | 289611 | 0.335 |
| 7273 | 59399 | | 343068 | 0.333 |
| 5278 | 39515 | ESTS, HIGHLY SIMILAR TO COFILIN, MUSCLE ISOFORM [MUS | 488362 | 0.332 |
| | | MUSCULUS] | | |
| 5166 | 3585 | P-SELECTIN GLYCOPROTEIN LIGAND 1 | 484838 | 0.331 |
| 7461 | 59620 | | 531428 | 0.329 |
| 2711 | 15537 | SORTING NEXIN 1 (SNX1) | 153995 | 0.328 |
| 5154 | 2804 | KERATIN 6 ISOFORM K6E (KRT6E) | 472084 | 0.322 |
| 423 | 3733 | PITUITARY HOMEOBOX 1 | 627344 | 0.319 |
| 3588 | 183677 | 1 RECORD MOVED FROM CLUSTER 14871 | 244050 | 0.319 |
| 545 | 4661 | TENASCIN | 23532 | 0.318 |
| 2402 | 4831 | TRANSCRIPTIONAL REGULATOR ISGF3 GAMMA SUBUNIT | 138181 | 0.309 |
| 3655 | 2819 | KIAA0110 | 252258 | 0.306 |
| 5163 | 1310 | DIHYDROPYRIDINE-SENSITIVE L-TYPE, CALCIUM CHANNEL | 484731 | 0.305 |
| | | BETA-3 SUBUNIT | | |
| 5508 | 100993 | 1 RECORD MOVED FROM CLUSTER 5160 | 510412 | 0.304 |
| 986 | 33121 | HEVIN LIKE PROTEIN | 41629 | 0.304 |
| 1858 | 6350 | MYO-INOSITOL MONOPHOSPHATASE 2 | 113566 | 0.304 |
| 1371 | 1435 | DYSTROGLYCAN | 70803 | 0.297 |
| 5625 | 172 | RIBOSOMAL PROTEIN L19 | 529388 | 0.294 |
| 6559 | 2772 | ISOVALERYL-COA DEHYDROGENASE | 756073 | 0.291 |
| 2985 | 639 | N-ACETYLGLUCOSAMINYLTRANSFERASE III | 178468 | 0.289 |
| 698 | 1288 | DEVELOPMENTALLY REGULATED GTP-BINDING PROTEIN | 29328 | 0.284 |
| | | DRG | | |
| 7017 | 135729 | 1 RECORD MOVED FROM CLUSTER 44399 | 193496 | 0.283 |
| 1234 | 139105 | 1 RECORD MOVED FROM CLUSTER 56868 | 50903 | 0.277 |
| 615 | 16397 | CLONE 23575 | 26451 | 0.271 |
| 6728 | 3917 | PROPIONYL-COA CARBOXYLASE ALPHA CHAIN | 109986 | 0.271 |
| 2212 | 1497 | ENOYL-COA HYDRATASE, MITOCHONDRIAL | 128447 | 0.269 |
| 4765 | 31344 | | 362530 | 0.268 |
| 6190 | 3429 | NF-AT4C | 625934 | 0.268 |
| 2612 | 1485 | ENDOTHELIAL TRANSCRIPTION FACTOR GATA-2 | 149809 | 0.264 |
| 622 | 2915 | LEUKOCYTE ANTIGEN CD37 | 26202 | 0.262 |
| 7294 | 56742 | | 346081 | 0.26 |
| 548 | 16381 | CLONE 23760 | 23760 | 0.253 |
| 5084 | 18785 | RAD1 | 470124 | 0.25 |
| 3943 | 11984 | CDC2-RELATED PROTEIN KINASE CHED | 271662 | 0.25 |
| 2517 | 3891 | COLLAGEN ALPHA1 (I) | 143925 | 0.25 |
| 1367 | 2381 | HYPOTHETICAL 33.4 KD PROTEIN | 70627 | 0.249 |
| 7095 | 44656 | ESTS | 261519 | 0.249 |
| 4758 | 33082 | SERINE/THREONINE PROTEIN KINASE | 362359 | 0.239 |
| 7371 | 215 | 8-OXOGUANINE DNA GLYCOSYLASE HOMOLOG 1 | 429057 | 0.236 |
| 4837 | 1862 | GLUTAMATE RECEPTOR 7 | 381812 | 0.235 |
| 4749 | 960 | CGMP-GATED CATION CHANNEL PROTEIN | 362167 | 0.233 |
| 1181 | 54049 | ESTS | 48418 | 0.231 |
| 4188 | 10671 | P162 | 293940 | 0.231 |
| 6441 | 4917 | TUBULIN BETA-1 CHAIN | 727352 | 0.231 |
| 649 | 717 | BRANCHED-CHAIN AMINO ACID AMINOTRANSFERASE, | 27710 | 0.227 |
| | | MITOCHONDRIAL | | |
| 6958 | 39960 | ESTS | 51015 | 0.225 |
| 4333 | 15747 | SER-THR PROTEIN KINASE PK428 | 302177 | 0.225 |
| 876 | 15557 | LGN PROTEIN | 36381 | 0.223 |
| 7621 | 566 | ATP-DEPENDENT RNA HELICASE A | 30626 | 0.206 |
| 486 | 56048 | ESTS, HIGHLY SIMILAR TO DNA-BINDING PROTEIN SATB1 | 21969 | 0.199 |
| | | [HOMO SAPIENS] | | |
| 824 | 2618 | INHIBITOR OF APOPTOSIS PROTEIN 2 | 34852 | 0.185 |
| 7174 | 32282 | ESTS | 294248 | 0.178 |
| 2319 | 34853 | ESTS, WEAKLY SIMILAR TO RNA-BINDING PROTEIN PIPPIN | 133479 | 0.17 |
| | | [R. NORVEGICUS] | | |
| 2014 | 1214 | CYTOCHROME P450 IVA11 | 120466 | 0.17 |
| 4628 | 3042 | MATRILYSIN | 338835 | 0.168 |
| 7158 | 6348 | PROTEASE M; neurosin | 283418 | 0.16 |
| 3033 | 138644 | 1 RECORD MOVED FROM CLUSTER 56351 | 184256 | 0.152 |
| 4296 | 131835 | 1 RECORD MOVED FROM CLUSTER 29918 | 300611 | 0.149 |
| 6512 | 505 | AQUAPORIN 5 | 741914 | 0.137 |
| 7106 | 39922 | ESTS | 264336 | 0.136 |
| 7261 | 872 | CASPASE-5 | 341763 | 0.131 |
| 6950 | 3655 | PEPTIDYL-PROLYL CIS-TRANS ISOMERASE, MITOCHONDRIAL | 49281 | 0.109 |
| 7456 | 56908 | BETA-DEFENSIN 1 | 527061 | 0.105 |
| 163 | 1846 | GLUCOSE-6-PHOSPHATE 1-DEHYDROGENASE | 32644 | 0.099 |
| 6104 | 4707 | THYMOSIN BETA-4 | 594922 | 0.094 |
| 7191 | 1890 | GLYCEROL KINASE | 305572 | 0.046 |
| 2859 | 5875 | IMMUNOGLOBULIN LIGHT CHAIN | 162999 | 0.03 |
| 1032 | 4710 | ERBA RELATED PROTEIN EAR-1 | 42706 | 0.015 |

What is claimed is:

1. A method for predicting the propensity for baldness, said method comprising the step of detecting the overexpression or the underexpression of a baldness-associated molecule of interest according to Table 1 in a subject, wherein the overexpression or the underexpression of said molecule is indicative of a propensity for baldness.

2. The method of claim 1, wherein overexpression of said molecule is indicative of a propensity for baldness and wherein said molecule is overexpressed in said subject.

3. The method of claim 1, wherein underexpression of said molecule is indicative of a propensity for baldness and wherein said molecule is underexpressed in said subject.

4. The method of claim 1, said method comprising detecting a baldness-associated mRNA.

5. The method of claim 1, said method comprising detecting a baldness-associated protein.

6. The method of claim 5, said method comprising detecting said baldness-associated protein in an immunoassay.

7. A method for identifying a modulator of hair loss, said method comprising the steps of:
(a) culturing a cell in the presence of said modulator to form a first cell culture;
(b) contacting RNA or cDNA from said first cell culture with a probe which comprises a polynucleotide sequence that encodes a baldness-associated protein selected from the group consisting of the polynucleotide sequences set forth in Table 1;
(c) determining whether the amount of probe that hybridizes to the RNA or cDNA from said first cell culture is increased or decreased relative to the amount of the probe that hybridizes to RNA or cDNA from a second cell culture grown in the absence of said modulator.

8. The method of claim 7, wherein said first and second cell cultures are obtained from a scalp cell.

9. A method for inhibiting the development of baldness, said method comprising the steps of introducing into a cell a baldness-associated molecule according to Table 1, wherein underexpression of said baldness-associated molecule is indicative of a propensity for baldness.

10. The method of claim 9, wherein said baldness-associated molecule is a nucleic acid encoding a baldness-associated protein.

11. The method of claim 10, wherein said baldness-associated molecule is a protein.

12. A method for reversing baldness, said method comprising the steps of introducing into a cell a baldness-associated molecule according to Table 1, wherein underexpression of said baldness-associated molecule is indicative of a propensity for baldness.

13. The method of claim 12, wherein said baldness-associated molecule is a nucleic acid encoding a baldness-associated protein.

14. The method of claim 12, wherein said baldness-associated molecule is a protein.

15. A method for inhibiting the development of baldness, said method comprising the steps of inhibiting in a cell overexpression of a baldness-associated molecule according to Table 1, wherein overexpression of said baldness-associated molecule is indicative of a propensity for baldness.

16. The method of claim 15, wherein said baldness-associated molecule is a nucleic acid that is inhibited using an antisense polynucleotide.

17. The method of claim 15, wherein said baldness-associated molecule is a protein that is inhibited using an antibody that specifically binds to the baldness-associated protein.

18. A method for reversing baldness, said method comprising the steps of inhibiting in a cell a baldness-associated molecule according to Table 1, wherein overexpression of said baldness-associated molecule is indicative of a propensity for baldness.

19. The method of claim 19, wherein said baldness-associated molecule a protein that is inhibited using an antibody that specifically binds to the baldness-associated protein.

20. The method of claim 19, wherein said baldness-associated molecule is a nucleic acid that is inhibited using an antisense polynucleotide.

21. A method for inhibiting the development of baldness in a patient in need thereof, said method comprising the step of administering to said patient a compound that modulates hair loss.

22. A method for reversing baldness in a patient, said method comprising the step of administering to said patient a compound that modulates hair loss.

23. A kit for detecting whether a hair follicle is becoming dormant, said kit comprising:
(a) a probe which comprises a polynucleotide sequence according to Table 1, associated with baldness; and
(b) a label for detecting the presence of said probe.

24. A cosmetic composition for inhibiting baldness in a patient, said cosmetic composition comprising a compound that modulates hair loss.

25. The cosmetic composition of claim 24, wherein said composition is in a form selected from the group consisting of shampoos, conditioners, lotions, sprays, ointments, oils, and gels.
